SEQUENCE LISTING (27 sequences)

SEQ ID NO: 1 - DNA, 20 bp, Artificial Sequence
  synthetic primer for amplifying a 143 base pair amplicon within the promoter region of the Fragile Mental Retardation-1 (FMR-1) gene
gtcaccgcccttcagccttc

SEQ ID NO: 2 - DNA, 21 bp, Artificial Sequence
  synthetic primer for amplifying a 143 base pair amplicon within the promoter region of the Fragile Mental Retardation-1 (FMR-1) gene
gccccgccctctctcttcaag

SEQ ID NO: 3 - DNA, 245 bp, Artificial Sequence
  synthetic amplicon FMR1 A1 from Fragile Mental Retardation-1 (FMR-1) promoter region
agagaggccgaactgggataaccggatgcatttgatttcccacgccactgagtgcacctctgcagaaatgggcgttctggccctcgcgaggcagtgcgacctgtcaccgcccttcagccttcccgccctccaccaagcccgcgcacgcccggcccgcgcgtctgtctttcgacccggcaccccggccggttcccagcagcgcgcatgcgcgcgctcccaggccacttgaagagagagggcggggc

SEQ ID NO: 4 - DNA, 20 bp, Artificial Sequence
  synthetic primer for amplifying a 143 base pair amplicon (FMR1 A2) of the Fragile Mental Retardation-1 (FMR-1) locus that lies downstream of the CCG repeat region
caccaaatcacaatggcaac

SEQ ID NO: 5 - DNA, 19 bp, Artificial Sequence
  synthetic primer for amplifying a 143 base pair amplicon (FMR1 A2) of the Fragile Mental Retardation-1 (FMR-1) locus that lies downstream of the CCG repeat region
gtagcagcgctgctaatgg

SEQ ID NO: 6 - DNA, 215 bp, Artificial Sequence
  synthetic amplicon FMR1 A2 from the Fragile Mental Retardation-1 (FMR-1) locus that lies 681 base pairs downstream of the 3′ end of the CCG repeat region
caccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctac

SEQ ID NO: 7 - DNA, 21 bp, Artificial Sequence
  synthetic primer for amplifying a Fragile Mental Retardation-1 (FMR-1) locus region US1 approximately 1 Kb upstream of the CCG repeat region
ggtactaagttcaatgctggc

SEQ ID NO: 8 - DNA, 20 bp, Artificial Sequence
  synthetic primer for amplifying a Fragile Mental Retardation-1 (FMR-1) locus region US1 approximately 1 Kb upstream of the CCG repeat region
gatgcacctccttgcaaccc

SEQ ID NO: 9 - DNA, 248 bp, Artificial Sequence
  synthetic amplicon US1 from the Fragile Mental Retardation-1 (FMR-1) locus 986 base pairs upstream of the 5′ end of the CCG repeat region
ggtactaagttcaatgctggcagtcgtttcttctttttttttcttttctgttttgctcaccgatttctcgttagcacttagcacagtgtctggcacacgatagatgctccgtcaacttctcagttggataccagcatcccgaagggaacatggattaaggcagctataagcacggtgtaaaaacaggaataagaaaaagttgaggtttgtttcacagtggaatgtaaagggttgcaaggaggtgcatc

SEQ ID NO: 10 - DNA, 21 bp, Artificial Sequence
  synthetic primer for amplifying a Fragile Mental Retardation-1 (FMR-1) locus region US2 approximately 2 Kb upstream of the CCG repeat region
gcatactcggtagcaaactag

SEQ ID NO: 11 - DNA, 20 bp, Artificial Sequence
  synthetic primer for amplifying a Fragile Mental Retardation-1 (FMR-1) locus region US2 approximately 2 Kb upstream of the CCG repeat region
cagtccctacagggtccttg

SEQ ID NO: 12 - DNA, 279 bp, Artificial Sequence
  synthetic amplicon US2 from the Fragile Mental Retardation-1 (FMR-1) locus 1952 base pairs upstream of the 5′ end of the CCG repeat region
gcatactcggtagcaaactaggtgtacacacagcaatgtgggtatagctcaaaaacagactgttgagtaaaacagtgggaaatagagatttacagtccaataccatctctgtaaatgcaagaggcataaacaaaacattatctgtgttaaattatcaaggatctctatcgaacatattgcagcttgtgtctagaagaatgagagtggggatcgagaaagatgaggaaaaaataatataaacactataaaataatgtaaacaaggaccctgtagggactg

SEQ ID NO: 13 - DNA, 22 bp, Artificial Sequence
  synthetic primer for amplifying a Fragile Mental Retardation-1 (FMR-1) locus region US3 approximately 3 Kb upstream of the CCG repeat region
catgcactggaatgaagtgaag

SEQ ID NO: 14 - DNA, 20 bp, Artificial Sequence
  synthetic primer for amplifying a Fragile Mental Retardation-1 (FMR-1) locus region US3 approximately 3 Kb upstream of the CCG repeat region
cttcgctggtatccctgcag

SEQ ID NO: 15 - DNA, 256 bp, Artificial Sequence
  synthetic amplicon US3 from the Fragile Mental Retardation-1 (FMR-1) locus 2984 base pairs upstream of the 5′ end of the CCG repeat region
catgcactggaatgaagtgaagggggaaagtagaaggtgagatagcggagataacagaagtcagagtatatagggcactgaagccagaataaataatgactctggcttttactctgagagatgtagggagtcactgggggattgtgagtggcagagtgacttgattgacacctcaacagggtcattctgtcttctgtgctgagaactgtcagagagggtacaacagtggacagtgactgcagggataccagcgaag

SEQ ID NO: 16 - DNA, 21 bp, Artificial Sequence
  synthetic primer for amplifying a Fragile Mental Retardation-1 (FMR-1) locus region US4 approximately 4 Kb upstream of the CCG repeat region
ctgctgacactgtaatggtgg

SEQ ID NO: 17 - DNA, 22 bp, Artificial Sequence
  synthetic primer for amplifying a Fragile Mental Retardation-1 (FMR-1) locus region US4 approximately 4 Kb upstream of the CCG repeat region
ctttcttgacactttctctgac

SEQ ID NO: 18 - DNA, 342 bp, Artificial Sequence
  synthetic amplicon US4 from the Fragile Mental Retardation-1 (FMR-1) locus 3904 base pairs upstream of the 5′ end of the CCG repeat region
ctgctgacactgtaatggtggatatgttatgcttttttcaaaacccacagagctatgcaacacaaagagtgaatcttaatgtaaactatagactttagttagtaataatgcatcagttgtaataaatgtacaatgcgaatgcaagatgttaataacaggaggaactgtaggggaggagggagattaggaagtacatagaaactctctccactatctgcttaatttttccataaatgtaaaactgttctaaaaaataaagccaattaattattctttttctaatatctcatttgaatgagaggtcatggttaaaggaaattgtcagagaaagtgtcaagaaag

SEQ ID NO: 19 - DNA, 20 bp, Artificial Sequence
  synthetic primer for amplifying a Fragile Mental Retardation-1 (FMR-1) locus region US5 approximately 4 Kb upstream of the CCG repeat region
gacaagccactgtctgggag

SEQ ID NO: 20 - DNA, 20 bp, Artificial Sequence
  synthetic primer for amplifying a Fragile Mental Retardation-1 (FMR-1) locus region US5 approximately 4 Kb upstream of the CCG repeat region
gacatatggcattgggcatc

SEQ ID NO: 21 - DNA, 191 bp, Artificial Sequence
  synthetic amplicon US5 from the Fragile Mental Retardation-1 (FMR-1) locus 4886 base pairs upstream of the 5′ end of the CCG repeat region
gacaagccactgtctgggagaaaatatctgcaaaatacataatctgataaagggctgatatctaaaatatataaagaacactgaaaaatcatcaataaaaaaaacccaatgggtaacagatctgagcagacaccttatcaaagaagatacatacgtgacaaacaagcataagatgcccaatgccatatgtc

SEQ ID NO: 22 - DNA, 20 bp, Artificial Sequence
  synthetic primer for amplifying a 165 base pair amplicon upstream of the dystrophia myotonica protein kinase (DMPK) gene CTG repeat region
gccaatgacgagttcggacg

SEQ ID NO: 23 - DNA, 19 bp, Artificial Sequence
  synthetic primer for amplifying a 165 base pair amplicon upstream of the dystrophia myotonica protein kinase (DMPK) gene CTG repeat region
gaggtcaacacccggcatg

SEQ ID NO: 24 - DNA, 165 bp, Artificial Sequence
  synthetic amplicon DMPK A1 from the dystrophia myotonica protein kinase (DMPK) locus 391 base pairs upstream of the 5′ end of the CCG repeat region
gccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctc

SEQ ID NO: 25 - DNA, 20 bp, Artificial Sequence
  synthetic primer for amplify a 319 base pair amplicon (FXN A1) of the frataxin (Friedrich′s ataxia) locus that lies upstream of the GAA repeat region
gaatggctgtggggatgagg

SEQ ID NO: 26 - DNA, 23 bp, Artificial Sequence
  synthetic primer for amplify a 319 base pair amplicon (FXN A1) of the frataxin (Friedrich′s ataxia) locus that lies upstream of the GAA repeat region
cttttaagcactggcaaccaatc

SEQ ID NO: 27 - DNA, 319 bp, Artificial Sequence
  synthetic amplicon FXN A1 from the frataxin (Friedrich′s ataxia) locus 170 base pairs upstream of the 5′ end of the GAA repeat region
gaatggctgtggggatgaggaagattcctcaaggggaggacatggtatttaatgagggtcttgaagatgccaaggaagtggtagagggtgtttcacgaggagggaaccgtctgggcaaaggccaggaaggcggaaggggatcccttcagagtggctggtacgccgcatgtattaggggagatgaaagaggcaggccacgtccaagccatatttgtgttgctctccggagtttgtactttaggcttgaacttcccacacgtgttatttggcccacattgtgtttgaagaaactttgggattggttgccagtgcttaaaag

(19) United States
(12) Patent Application Publication
(10) Pub. No.: US 2013/0316339 A1
(43) Pub. Date: Nov. 28, 2013

(54) DETECTION OF NUCLEIC ACID SEQUENCES ADJACENT TO REPEATED SEQUENCES

(71) Applicants: Jared Ordway, St. Louis, MO (US); Blaire Bacher, St. Louis, MO (US)

(72) Inventors: Jared Ordway, St. Louis, MO (US); Blaire Bacher, St. Louis, MO (US)

(73) Assignee: ORION GENOMICS LLC, St. Louis, MO (US)

(21) Appl. No.: 13/819,805

(22) PCT Filed: Aug. 31, 2011
(86) PCT No.: PCT/US2011/050063

§ 371 Date: Aug. 15, 2013

Related U.S. Application Data
(60) Provisional application No. 61/379,197, filed on Sep. 1, 2010.

(51) Int. Cl. C12Q 1/68

(52) U.S. Cl. CPC C12Q 1/6858; C12Q 1/6883
USPC 435/6.11; 435/6.12

(57) ABSTRACT

The present invention provides methods of quantifying a target locus adjacent to an extended repeat sequence in genomic DNA. The present invention further provides methods and kits for detecting methylation at a target locus adjacent to an extended repeat sequence in genomic DNA.

### CROSS-REFERENCES TO RELATED APPLICATIONS

This application claims the benefit of U.S. Provisional Application No. 61/379,197, filed Sep. 1, 2010. This priority application is hereby incorporated in its entirety

### BACKGROUND OF THE INVENTION

Expanded repeat sequences, an increase in the number of copies of a short nucleotide sequence in genomic DNA, are linked to a number of human diseases. In one category of expanded repeat-associated diseases, the repeated sequence is located within an untranslated region of a gene and disrupts processes such as gene expression in the cell. In another category of diseases, expanded repeat sequences are translated into expanded polyglutamine tracts, resulting in protein aggregations that are toxic to the cell.

For some expanded repeat diseases, such as fragile X syndrome, regions of the gene associated with the expanded repeat sequence are further characterized by hypermethylation. In mutated alleles of the Fragile Mental Retardation-1 (FMR-1) gene associated with fragile X syndrome (alleles having >200 copies of a CGG repeat), the promoter region of the FMR-1 gene, including but not limited to the CGG repeat sequence, is hypermethylated, shutting down gene transcription. In contrast, in normal and premutation-sized alleles (alleles having a smaller number of copies of the CGG repeat), the promoter region of FMR-1 is unmethylated or less highly methylated. Thus, for diseases associated with expanded repeat sequences and further characterized by increased methylation, detection of altered methylation profiles at loci where such alterations are associated with disease can be used to provide diagnoses or prognoses of disease.

### BRIEF SUMMARY OF THE INVENTION

The present invention provides for a method of quantifying a target locus adjacent to a repeated sequence in genomic DNA, wherein proximity to the repeated sequence interferes with amplification of the target locus. In some embodiments, the method comprises cleaving the genomic DNA with a first restriction enzyme between the target locus and the repeated sequence, wherein the cleaving does not cleave the target locus; and quantitatively amplifying the target locus.

In some embodiments, the target locus is at least 100, 200, 300, 400, or 500 base pairs long.

In some embodiments, the quantitative amplifying comprises quantitative polymerase chain reaction (qPCR).

In some embodiments, the repeated sequence comprises a continuous repetition of a motif of two, three, four, or five base pairs repeated at least ten times. In some embodiments, the repeated sequence comprises a continuous repetition of a motif of three base pairs repeated at least ten times. In some embodiments, the repeated sequence comprises a continuous repetition of a motif of three base pairs repeated at least forty times.

In some embodiments, the repeated sequence is selected from the group consisting of CAG, CTG, CCG, CGG, GAC, GTC, GGC, GCC, GCG, ACG, TCG, CGA, CGT, CGC, GCA, GCT, GCC, TGC, AGC and CA.

In some embodiments, the repeated sequence and the target locus are less than 10 kb (e.g., less than 8, 6, 4, or 2 kb) apart.

In some embodiments, the genomic DNA comprises human DNA.

In some embodiments, the target locus is a promoter region.

In some embodiments, the target locus is within the human Fragile Mental Retardation-1 (FMR-1); Fragile Mental Retardation-2 (FMR-2); dystrophia myotonica protein kinase (DMPK); spinocerebellar ataxia type 8 (SCA8); androgen receptor (AR); huntingtin (IT15); dentatorubralpallidoluysian atrophy (DRPLA); spinocerebellar ataxia type 1 (SCA1); spinocerebellar ataxia type 2 (SCA2); spinocerebellar ataxia type 3 (SCA3); spinocerebellar ataxia type 6 (SCA6); spinocerebellar ataxia type 7 (SCA7); or cystatin B (CSTB) gene.

In some embodiments, the first restriction enzyme is a methylation-insensitive restriction enzyme.

In some embodiments, the method further comprising contacting the DNA, before, concurrent with, or after the cleaving, but before the quantitative amplifying, with at least one more restriction enzyme.

In some embodiments, the at least one more restriction enzyme is a methylation-dependent restriction enzyme or a methylation-sensitive restriction enzyme. In some embodiments, the methylation-dependent restriction enzyme or the methylation-sensitive restriction enzyme are used under conditions such that the amount of remaining intact copies of the locus is inversely proportional or directly proportional, respectively, to the methylation density of the locus. In some embodiments, the method comprises contacting the DNA with a methylation-dependent restriction enzyme or a methylation-sensitive restriction enzyme, before, concurrent with, or after cleaving with a first restriction enzyme, but before the quantitative amplification step.

In some embodiments, the first restriction enzyme has a four, six, or eight nucleotide recognition sequence.

In some embodiments, the first restriction enzyme is not AluI.

In some embodiments, the DNA is cleaved with the first restriction enzyme for more than 1 hour.

The present invention also provides for methods of detecting the quantity of methylation at a target locus adjacent to a repeated sequence in a genomic DNA sample, wherein proximity to the repeated sequence interferes with amplification of the target locus. In some embodiments, the method comprises:

(a) cleaving the genomic DNA sample with a first restriction enzyme between the target locus and the repeated sequence, wherein the cleaving does not cleave the target locus;
(b) dividing the genomic DNA sample into at least two physically distinct portions, thereby generating a first portion and a second portion;
(c) contacting the first portion with a methylation-sensitive restriction enzyme to obtain a genomic DNA sample comprising fragmented unmethylated copies of the target locus and intact methylated copies of the target locus;
(d) quantifying the number of intact copies of the target locus in the first portion by quantitative amplification;
(e) contacting the second portion with a methylation-dependent restriction enzyme to obtain a genomic DNA sample comprising fragmented methylated copies of the target locus and intact unmethylated copies of the target locus;
(f) quantifying the number of intact copies of the target locus in the second portion by quantitative amplification; and
(g) determining the quantity of methylation at the target locus by comparing the number of intact copies of the target locus in the first portion and the number of intact copies of the target locus in the second portion.

In some embodiments, the dividing comprises dividing the sample into at least three portions, wherein the third portion is not cleaved with a restriction enzyme other than the first restriction enzyme, and the method further comprises quantifying the number of intact copies of the target locus in a third portion by quantitative amplification; and comparing the number of intact copies of the target locus in the third portion to the number of intact copies of the target locus in the first portion and/or the number of intact copies of the target locus in the second portion.

In some embodiments, the dividing comprises dividing the sample into at least four portions, wherein the third portion is not cleaved with a restriction enzyme other than the first restriction enzyme, and the method further comprises:

contacting a fourth portion with a methylation-sensitive restriction enzyme and a methylation-dependent restriction enzyme;
quantifying the number of intact copies of the target locus in the fourth portion by quantitative amplification; and
determining the quantity of methylation at the target locus by comparing the number of intact copies of the target locus in the fourth portion to the number of intact copies of the target locus in the third portion and/or the number of intact copies of the target locus in the first portion and/or the number of intact copies of the target locus in the second portion.

In some embodiments, the first restriction enzyme is a methylation-insensitive restriction enzyme.

In some embodiments, the quantitative amplifying comprises quantitative polymerase chain reaction (qPCR).

In some embodiments, the repeated sequence comprises a continuous repetition of a motif of two, three, four, or five base pairs repeated at least ten times.

In some embodiments, the repeated sequence comprises a continuous repetition of a motif of three base pairs repeated at least ten times.

In some embodiments, the repeated sequence comprises a continuous repetition of a motif of three base pairs repeated at least forty times.

In some embodiments, the repeated sequence is selected from the group consisting of CAG, CTG, CCG, CGG, GAC, GTC, GGC, GCC, GCG, ACG, TCG, CGA, CGT, CGC, GCA, GCT, GCC, TGC, AGC and CA.

In some embodiments, the repeated sequence and the target locus are less than 10 kb (e.g., less than 8, 6, 4, or 2 kb) apart.

In some embodiments, the genomic DNA is human DNA.

In some embodiments, the target locus is a promoter region.

In some embodiments, the target locus is within the human Fragile Mental Retardation-1 (FMR-1); Fragile Mental Retardation-2 (FMR-2); dystrophia myotonica protein kinase (DMPK); spinocerebellar ataxia type 8 (SCA8); androgen receptor (AR); huntingtin (IT15); dentatorubralpallidoluysian atrophy (DRPLA); spinocerebellar ataxia type 1 (SCA1); spinocerebellar ataxia type 2 (SCA2); spinocerebellar ataxia type 3 (SCA3); spinocerebellar ataxia type 6 (SCA6); spinocerebellar ataxia type 7 (SCA7); or cystatin B (CSTB) gene.

The present invention also provides for a kit for quantifying a target locus adjacent to a repeated sequence. In some embodiments, the kit comprises:

(ii) one or more oligonucleotides that specifically amplify the target locus of the gene; and
(iii) a restriction enzyme (including but not limited to a methylation-insensitive restriction enzyme) that cleaves human genomic DNA between the repeated sequence and the target locus but does not cleave the target locus.

In some embodiments, the target locus is a promoter region.

In some embodiments, the target locus is within the human Fragile Mental Retardation-1 (FMR-1); Fragile Mental Retardation-2 (FMR-2); dystrophia myotonica protein kinase (DMPK); spinocerebellar ataxia type 8 (SCA8); androgen receptor (AR); huntingtin (IT15); dentatorubralpallidoluysian atrophy (DRPLA); spinocerebellar ataxia type 1 (SCA1); spinocerebellar ataxia type 2 (SCA2); spinocerebellar ataxia type 3 (SCA3); spinocerebellar ataxia type 6 (SCA6); spinocerebellar ataxia type 7 (SCA7); or cystatin B (CSTB) gene.

In some embodiments, the kit further comprises a methylation-dependent restriction enzyme and/or a methylation-sensitive restriction enzyme.

In some embodiments, the kit comprises a methylation-dependent restriction enzyme and a methylation-sensitive restriction enzyme.

In some embodiments, the kit further comprises reagents for detecting the amplification of the target locus by quantitative polymerase chain reaction (qPCR).

### DEFINITIONS

The term “repeated sequence” or “expanded repeat sequence,” as used herein, refers to a polynucleotide sequence having repeated motifs of short (e.g., 2 bp, 3 bp, 4 bp, 5 bp, 6 bp, 7 bp, 8 bp, 9 bp, 10 bp, 11 bp, 12 bp, 13 bp, 14 bp, or 15 bp) nucleotide sequences. In some embodiments, the motifs in the repeated sequence are continuously repeated (i.e., there are no intervening nucleotides between adjacent motifs). In some embodiments, the nucleotide motifs are repeated at least 5, 10, 15, 20, 25, 30, 35, 40, 45, 50, 60, 70, 80, 90, 100, 200, 300, 400, 500, 600, 700, 800, 900, 1000 times or more in the polynucleotide sequence. Expanded repeat sequences, including but not limited to dinucleotide repeats and trinucleotide repeats, have been reported as being linked to various diseases, such as neurodegenerative diseases (e.g., Huntington's disease, spinobulbar muscular atrophy, and spinocerebellar ataxia), Fragile X syndrome, Friedreich's ataxia, and myotonic dystrophy.

The term “target locus” refers to a target (i.e., to be detected) polynucleotide sequence within a population of nucleic acids (e.g., a genome). In some embodiments, a target locus refers to a target sequence within a gene, or a segment of DNA involved in producing a polypeptide chain. A gene can include, without limitation, a coding region comprising intervening sequences (introns) between discrete coding segments (exons), as well as regions preceding and following the coding region, such as a promoter region and a 3′-untranslated region.

The term “promoter region” refers to a polynucleotide sequence that initiates and facilitates the transcription of a target gene sequence in the presence of RNA polymerase and transcription regulators. A promoter region is located 5′ to the transcribed gene and, as used herein, includes the sequence upstream of the transcription start site. Typically, the promoter region comprises the polynucleotide region that is 1-4 kb upstream (5′) of the transcriptional start site, e.g., within 500 bp, 1000 bp, 1500 bp, or 2000 bp of the transcriptional start site, though the promoter region can be larger in some genes. In some embodiments, the promoter region comprises the polynucleotide sequence that is within 500 bp, 1000 bp, 1500 bp, 2000 bp, 3000 bp, 4000 bp, 5000 bp, 6000 bp, 7000 bp, 8000 bp, 9000 bp or 100000 bp of the transcription start site.

The term “adjacent to a repeated sequence,” as used to describe the proximity of a target locus to a repeated sequence, refers to a target locus that is less than 10 kb, e.g., less than 9 kb, less than 8 kb, less than 7 kb, less than 6 kb, less than 5 kb, less than 4 kb, less than 3 kb, less than 2 kb, less than 1 kb, less than 900 bp, less than 800 bp, less than 700 bp, less than 600 bp, less than 500 bp, less than 400 bp, less than 300 bp, less than 200 bp, or less than 100 bp away from the beginning or end of a repeated sequence. “Proximity” is used herein synonymously with “adjacent to.”

A repeated sequence “interferes with amplification” of a target locus that is in proximity to the repeated sequence when the magnitude of amplification of the target sequence demonstrates substantial variability when linked to the repeated sequence compared to when the target sequence is not linked to the repeated sequence. For example, when measured using quantitative PCR, a coefficient of variance between replicates of at least 20, 30, 40% or more is considered significant variability.

The term “restriction enzyme” refers to an enzyme that cuts a nucleic acid sequence (e.g., DNA) at specific recognition nucleotide sequences, or “restriction sites.”

The term “methylation-insensitive restriction enzyme” refers to a restriction enzyme that cuts DNA regardless of the methylation state of the base of interest (A or C) at or near the recognition sequence of the enzyme.

The term “methylation-sensitive restriction enzyme” refers to a restriction enzyme (e.g., PstI) that cleaves at or in proximity to an unmethylated recognition sequence but does not cleave at or in proximity to the same sequence when the recognition sequence is methylated. Generally, use of “methylation” in the context of this invention relates to methylcytosine methylation unless specifically indicated otherwise. Exemplary methylation sensitive restriction enzymes are described in, e.g., McClelland et al, Nucleic Acids Res. 22(17):3640-59 (1994) and on the world wide web at rebase.neb.com. Suitable methylation-sensitive restriction enzymes that do not cleave at or near their recognition sequence when a cytosine within the recognition sequence is methylated at position C⁵ include, e.g., AatII, Acc65I, AccI, AciI, AclI, AfeI, AgeI, AhdI, AleI, ApaI, ApaLI, AsaI, AsiSI, AvaI, AvaII, BaeI, BanI, BbvCI, BbeI, BceI, BcgI, BcoDI, BfuAI, BfuCI, BglI, BmgBI, BsaI, BsaI, BsaBI, BsaHI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, BtgZI, Cac8I, ClaI, DpnI, DraIII, DrdI, EaeI, EagI, EarI, EciI, EcoRI, EcoRV, FauI, Fnu4HI, FokI, FseI, FspI, HaeII, HgaI, HhaI, HinP1I, HinfI, HincII, HpaI, HpaII, Hpy166II, Hyp188III, Hpy99I, HpyAV, HpyCH4IV, KasI, MboI, MluI, MapA1I, MmeI, MspI, MspA1I, MwoI, NaeI, NarI, NciI, NheI, NlaIV, NotI, NgoMIV, NruI, Nt.BbvCI, Nt.BsmAI, Nt.CviPII, PaeR7I, PhoI, PleI, PmeI, PmlI, PshAI, PspOMI, PvuI, RsaI, RsrII, SacII, SalI, Sau3AI, Sau96I, ScrFI, SfaNI, SfiI, SfoI, SgrAI, SmaI, SnaBI, StyD4I, TfiI, TseI, TspMI, XmaI, and ZraI. Suitable methylation-sensitive restriction enzymes that do not cleave at or near their recognition sequence when an adenosine within the recognition sequence is methylated at position N⁶ include, e.g., Mbo I. One of skill in the art will appreciate that homologs and orthologs of the restriction enzymes described herein are also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence.

The term “methylation-dependent restriction enzyme” refers to a restriction enzyme that cleaves at or near a methylated recognition sequence, but does not cleave at or near the same sequence when the recognition sequence is not methylated. Generally, use of “methylation” in the context of this invention relates to methylcytosine methylation unless specifically indicated otherwise. Methylation-dependent restriction enzymes include those that cut at a methylated recognition sequence (e.g., DpnI) and enzymes that cut at a sequence that is not at the recognition sequence (e.g., McrBC). For example, McrBC requires two half-sites. Each half-site must be a purine followed by 5-methyl-cytosine (R5mC) and the two half-sites must be no closer than 20 base pairs and no farther than 4000 base pairs away from each other (N20-4000). McrBC generally cuts close to one half-site or the other, but cleavage positions are typically distributed over several base pairs approximately 32 base pairs from the methylated base. Exemplary methylation-dependent restriction enzymes include, e.g., McrBC (see, e.g., U.S. Pat. No. 5,405,760), McrA, MrrA, Dpn I, Msp JI, Lpn PI and Fsp IE. One of skill in the art will appreciate that homologs and orthologs of the restriction enzymes described herein are also suitable for use in the present invention.

As used herein, the term “recognition sequence” refers to a primary nucleic acid sequence at which a restriction enzyme cleaves, or that must be present for the restriction enzyme to cleave in situations in which the restriction enzyme cleaves outside its recognition sequence, and does not reflect the methylation status of the sequence.

The term “methylation” refers to cytosine methylation at C5 or N4 positions of cytosine (“5mC” and “4mC,” respectively), at the N6 position of adenine (“6mA”), or other types of nucleic acid methylation. Aberrant methylation of a DNA sequence (i.e., hypermethylation or hypomethylation) may be associated with a disease, condition or phenotype (e.g., cancer, vascular disease, cognitive disorders, or other epigenetic trait). An “unmethylated” DNA sequence contains substantially no methylated residues at least at recognition sequences for a particular methylation-dependent or methylation-sensitive restriction enzyme used to evaluate the DNA. “Methylated” DNA contains methylated residues at least at the recognition sequences for a particular methylation-dependent or methylation-sensitive restriction enzyme used to evaluate the DNA. It is understood that while a DNA sequence referred to as “unmethylated” may generally have substantially no methylated nucleotides along its entire length, the definition encompasses nucleic acid sequences that have methylated nucleotides at positions other than the recognition sequences for restriction enzymes. Likewise, it is understood that while a DNA sequence referred to as “methylated” may generally have methylated nucleotides along its entire length, the definition encompasses nucleic acid sequences that have unmethylated nucleotides at positions other than the recognition sequences for restriction enzymes. “Hemimethylated” DNA refers to double stranded DNA in which one strand of DNA is methylated at a particular locus and the other strand is unmethylated at that particular locus.

The term “dividing” or “divided,” in the context of dividing DNA, typically refers to dividing a population of nucleic acids (e.g., genomic DNA) isolated from a sample into two or more physically distinct portions, each of which comprise all of the sequences present in the sample.

As used herein, a “partial digestion” refers to contacting DNA with a restriction enzyme under appropriate reaction conditions such that the restriction enzyme cleaves some but not all of possible cleavage sites for that particular restriction enzyme in the DNA. A partial digestion of the sequence can be achieved, e.g., by contacting DNA with an active restriction enzyme for a shorter period of time than is necessary to achieve a complete digestion and then terminating the reaction, by contacting DNA with less active restriction enzyme than is necessary to achieve complete digestion with a set time period (e.g., 30, 60, 90, 120, 150, 150, or 240 minutes), or under other altered reaction conditions that allow for the desired amount of partial digestion. In particular, with methylation-dependent restriction enzymes such as McrBC, an appropriate amount of enzyme for an appropriate time can be determined such that the amount remaining intact is inversely proportional to the amount of methylated recognition sequences present. See, e.g., WO2005/040399; WO 2005/042704; and Holeman et al., Biotechniques 43(5): 683-693 (2007).

The terms “amplify” or “amplification” refer to any chemical, including enzymatic, reaction that results in an increased number of copies of a template nucleic acid sequence or an increased signal indicating that the template nucleic acid is present in the sample. Amplification reactions include polymerase chain reaction (PCR) and ligase chain reaction (LCR) (see U.S. Pat. Nos. 4,683,195 and 4,683,202; PCR PROTOCOLS: A GUIDE TO METHODS AND APPLICATIONS (Innis et al., eds, 1990)), strand displacement amplification (SDA) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)); branched DNA signal amplification (bDNA) (see, e.g., Iqbal et al., Mol. Cell Probes 13(4):315-320 (1999)), and linear amplification. Amplifying includes, e.g., ligating adaptors that comprise T3 or T7 promoter sites to the template nucleic acid sequence and using T3 or T7 polymerases to amplify the template nucleic acid sequence.

The terms “quantitative amplifying,” “quantitatively amplifying,” and “quantitative amplification” refer to an amplification reaction in which the number of copies that is produced from the template nucleic acid sequence is quantitatively determined. In some embodiments, quantitative amplification is used to quantify the amount of intact DNA within a locus flanked by amplification primers following digestion with a restriction enzyme.

### BRIEF DESCRIPTION OF THE DRAWINGS

FIG. 1A. Map of the FMR1 locus. The positions of the FMR1 promoter, exon 1, and the CCG repeat region are indicated. The scale bar at the top of the diagram is in base pairs. The location of the qPCR amplicon tested in Tables 4 and 5 and FIGS. 4 and 5 is indicated as FMR1 A1.

FIG. 1B. Map of the FMR1 locus. The map shown in FIG. 1A is reproduced including the locations of all CpG dinucleotides within the locus.

FIG. 2. Strategy used for qPCR and Assay replicates. The assay strategy is diagrammed for a single DNA sample. Three assay replicates are performed (1_{—}1, 1_{—}2 and 1_{—}3). For each assay replicate, the sample is divided into four portions. The first portion is mock treated (UT), the second portion is digested with a methylation dependent restriction enzyme (for example, McrBC), the third portion is digested with a methylation sensitive restriction enzyme (for example, Hha I) and the fourth portion is digested with both the methylation dependent and the methylation sensitive restriction enzymes (Double), as described in the Examples. Each treatment is analyzed by three independent qPCR reactions using the indicated primer pairs (qPCR). Average, standard deviation and coefficient of variance are calculated for each qPCR triplicate and for each Assay triplicate as described in Tables 1, 2 and 3.

FIG. 3. Map of the FMR1 locus. The map shown in FIG. 1 is reproduced to indicate the sites cut by the methylation insensitive restriction enzyme Alu I.

FIG. 4A. Percentage of Densely Methylated Molecules measured by three FMR1 A1 Assay replicates for Samples 1, 2 and 3. Each point represents the average of three Assay measurements. The error bars represent the standard deviations of each Assay measurement. The samples in FIG. 4A were analyzed without secondary digestion with Alu I.

FIG. 4B. Percentage of Densely Methylated Molecules measured by three FMR1 A1 Assay replicates for Samples 1, 2 and 3 following secondary digestion by Alu I. Each point represents the average of three Assay measurements. The error bars represent the standard deviations of each Assay measurement.

FIG. 5. Average FMR1 A1 Ct values for untreated portions with and without secondary digestion with Alu I. Bars represent the average Ct value across 9 qPCR reactions. Error bars indicate the standard deviation across the same 9 qPCR replicates.

FIG. 6A. Average Ct values for untreated portions before (−) and after (+) secondary digestion with Bsa WI. The graph shows analysis of Sample 4 using amplicons US1, US2, US3, US4 and US5 separately. These amplicons are located approximately 1 Kb, 2 Kb, 3 Kb, 4 Kb and 5 Kb upstream of the CCG repeat region, respectively.

FIG. 6B. Average Ct values for untreated portions before (−) and after (+) secondary digestion with Bsa WI. The graph shows analysis of Sample 5 using amplicons US1, US2, US3, US4 and US5 separately. These amplicons are located approximately 1 Kb, 2 Kb, 3 Kb, 4 Kb and 5 Kb upstream of the CCG repeat region, respectively.

FIG. 7A. Average delta Ct values of the untreated portion without secondary digestion minus the untreated portion with secondary digestion. The graph shows the same data as in FIG. 6A, but plotted by delta Ct rather than absolute Ct value.

FIG. 7B. Average delta Ct values of the untreated portion without secondary digestion minus the untreated portion with secondary digestion. The graph shows the same data as in FIG. 6B, but plotted by delta Ct rather than absolute Ct value.

FIG. 8A. Percentage of Densely Methylated Molecules measured by three DMPK A1 Assay replicates for Sample 6. Each point represents the average of three Assay measurements. The error bars represent the standard deviations of each Assay measurement. Data are plotted for analysis without secondary digestion with Eco NI (−) and with secondary digestion with Eco NI (+).

FIG. 8B. Average DMPK A1 Ct values for untreated portions with and without secondary digestion with Eco NI. Bars represent the average Ct value across 9 qPCR reactions. Error bars indicate the standard deviation across the same 9 qPCR replicates.

FIG. 9A. Percentage of Densely Methylated Molecules measured by three FXN A1 Assay replicates for Sample 7. Each point represents the average of three Assay measurements. The error bars represent the standard deviations of each Assay measurement. Data are plotted for analysis without secondary digestion with Avr II (−) and with secondary digestion with Avr II (+).

FIG. 9B. Percentage of Densely Methylated Molecules measured by three FXN A1 Assay replicates for Sample 8. Each point represents the average of three Assay measurements. The error bars represent the standard deviations of each Assay measurement. Data are plotted for analysis without secondary digestion with Avr II (−) and with secondary digestion with Avr II (+).

FIG. 10. Average FXN A1 Ct values for untreated portions with and without secondary digestion with Avr II. Bars represent the average Ct value across 9 qPCR reactions. Error bars indicate the standard deviation across the same 9 qPCR replicates.

### DETAILED DESCRIPTION

### I. Introduction

The present invention provides methods for quantifying a target locus in genomic DNA that is located near an extended repeat sequence. It has been discovered that quantitative amplification of target DNA sequences near extended repeat sequences can yield inconsistent results. Surprisingly, cutting the genomic DNA between the target DNA sequence and the extended repeat sequence significantly improves the consistency and reliability of a subsequent quantitative amplification reaction. Without being bound to a particular theory, it is believed that the presence of an extended repeat sequence adjacent to the target locus to be amplified can interfere with the amplification reaction. Thus, in one aspect the present invention provides a method of quantifying a target locus adjacent to a repeated sequence in genomic DNA, wherein proximity to the repeated sequence interferes with amplification of the locus, the method comprising: cleaving the genomic DNA with a first restriction enzyme between the target locus and the repeated sequence, wherein the cleaving does not cleave the target locus; and quantitatively amplifying the target locus.

In some aspects, the methods of the present invention can be used in conjunction with methods of determining the presence of methylation at a target locus and quantifying the amount of methylation at a target locus. The presence and/or density (i.e., quantity) of methylation at a DNA locus can be useful for providing diagnoses and prognoses for various diseases, including various cancers, and methylation status can also be useful in providing diagnoses and prognoses for diseases characterized by expanded repeat sequences, such as fragile X syndrome.

### II. Target Loci

In one aspect, the present invention provides methods of amplifying a target locus that is adjacent to a repeated sequence in genomic DNA. In some embodiments, the target locus is located within a gene. As used herein, a gene includes coding regions (exons), introns, promoter sequence, 5′ untranslated sequence, and 3′ untranslated sequence. In some embodiments, the target locus is located within a gene that is characterized by the presence of expanded repeat sequences. Expanded repeat sequences, or repeated sequences, are continuous (e.g., directly repeated, without intervening nucleotides) repeated motifs of short nucleic acid sequences. In some embodiments, the repeated motif is a 2, 3, 4, 5, 6, 7, 8, 9, 10, 11, 12, 13, 14, or 15 nucleotide sequence. In some embodiments, the repeated motif is a trinucleotide (i.e., 3 nucleotide) sequence. In some embodiments, the trinucleotide repeat is a CTG motif; a CAG motif, a CCG motif, a CAG motif, a CGG motif, or a GAA motif. By convention, the sequence of the repeated motif is the sequence of the expressed strand rather than the sequence of the anti-sense strand.

Without intending to limit the scope of the invention, it is believed that the interfering repeated sequences form secondary structures that interfere with primer extension. Thus, in some embodiments, the interfering repeats are predicted to form an interfering secondary structure. In general, this may occur if the repeated sequence unit includes greater than 60% G+C content, for example: CAG, CTG, CCG, CGG, GAC, GTC, GGC, GCC, GCG, ACG, TCG, CGA, CGT, CGC, GCA, GCT, GCC, TGC, and AGC. Each of these repeat units could potentially form mismatched hairpin DNA structures in which the C bases of one strand base pair with the G bases of the same strand. DNA structures formed by repeated sequence units include Hy3-type intramolecular triplex, hairpin structures, quadruplex structures, cruciform structures, slipped strand structures, folded slipped structures and Z-DNA structures (as described in Sinden et al. (2002) J. Biosci. (Suppl. 1) 27: 53-65). It is understood that unusual secondary structures may for as a consequence of repeated sequences other than trinucleotide repeat sequences (for example, dinucleotide repeats or tetranucleotide repeats). For example, a polymorphic CA repeat in the first intron of epidermal growth factor receptor gene (EGFR) Bonus secondary structures that interfere with EGFR gene transcription in a CA repeat length-dependent manner (Gebhardt et al. (2000) Histol. Histopathol. 15(3): 929-36).

Expanded repeat sequences, whether within a region of a gene (e.g., within a promoter region or within an untranslated region), or not, may be associated with a disease phenotype, with diseased individuals having an increased number of repeats of a motif as compared to a normal (i.e., non-diseased) individual. For example, an expanded repeat sequence associated with a disease phenotype may comprise a motif (e.g., a repeated motif as described above) that is continuously repeated (i.e., directly repeated without any sequences other than the motif occurring between repeats) at least 10, at least 20, at least 30, at least 40, at least 50, at least 60, at least 70, at least 80, at least 90, at least 100, at least 150, at least 200, at least 300, at least 400, or at least 500 or more times.

Examples of genes that are characterized by the presence of expanded repeat sequences include, but are not limited to, Fragile Mental Retardation-1 (FMR-1, implicated in fragile X syndrome); Fragile Mental Retardation-2 (FMR-2, implicated in FRAXE mental retardation syndrome); dystrophia myotonica protein kinase (DMPK, implicated in myotonic dystrophy); spinocerebellar ataxia type 8 (SCA8, implicated in spinocerebellar ataxia type 8); frataxin (FXN, implicated in Friedrich's ataxia); androgen receptor (AR, implicated in spinobulbar muscular atrophy); huntingtin (IT15, implicated in Huntington's disease); dentatorubralpallidoluysian atrophy (DRPLA, implicated in dentatorubralpallidoluysian atrophy); spinocerebellar ataxia type 1 (SCA1, implicated in spinocerebellar ataxia type 1); spinocerebellar ataxia type 2 (SCA2, implicated in spinocerebellar ataxia type 2); spinocerebellar ataxia type 3 (SCA3, implicated in spinocerebellar ataxia type 3); spinocerebellar ataxia type 6 (SCA6, implicated in spinocerebellar ataxia type 6); spinocerebellar ataxia type 7 (SCA7, implicated in spinocerebellar ataxia type 7); and cystatin B (CSTB, implicated in progressive myoclonus epilepsy of Unverricht-Lundborg type). In some embodiments, the gene is a human gene. Thus, in some embodiments, a target locus to be amplified is within the human FMR-1, FMR-2, DMPK, SCA8, FXN, AR, IT15, DRPLA, SCA1, SCA2, SCA3, SCA6, SCA7, or CSTB gene.

In some embodiments, the target locus is a promoter region of a gene (e.g., FMR-1, FMR-2, DMPK, SCA8, FXN, AR, IT15, DRPLA, SCA1, SCA2, SCA3, SCA6, SCA7, or CSTB), for example, within 100 bp, 200 bp, 300 bp, 400 bp, 500 bp, 600 bp, 700 bp, 800 bp, 900, or 100 bp of a transcriptional start site. In some embodiments, the target locus is a coding region (exon) of a gene described herein (e.g., FMR-1, FMR-2, DMPK, SCA8, FXN, AR, IT15, DRPLA, SCA1, SCA2, SCA3, SCA6, SCA7, or CSTB). In some embodiments, the target locus is an intron of a gene described herein (e.g., FMR-1, FMR-2, DMPK, SCA8, FXN, AR, IT15, DRPLA, SCA1, SCA2, SCA3, SCA6, SCA7, or CSTB). In some embodiments, the target locus is an untranslated region (e.g., 5′ UTR or 3′ UTR) of a gene described herein (e.g., FMR-1, FMR-2, DMPK, SCA8, FXN, AR, IT15, DRPLA, SCA1, SCA2, SCA3, SCA6, SCA7, or CSTB). In some embodiments, the target locus spans multiple regions of a gene as described herein, for example, promoter sequence and 5′ untranslated sequence; 5′ untranslated sequence and exon sequence; exon sequence and intron sequence; exon sequence and 3′ untranslated sequence; 5′ untranslated sequence, exon sequence and intron sequence; promoter sequence, 5′ untranslated sequence, exon sequence and intron sequence; intron sequence, exon sequence, and 3′ untranslated sequence; or exon sequence and 3′ untranslated sequence.

In some embodiments, the target locus to be amplified is located within 10 kb of the beginning of the repeated sequence (if the target locus is upstream of the repeated sequence) or within 10 kb of the end of the repeated sequence (if the target locus is downstream of the repeated sequence). In some embodiments, the target locus to be amplified is within 10 kb, within 9 kb, within 8 kb, within 7 kb, within 6 kb, within 5 kb, within 4 kb, within 3 kb, within 2 kb, within 1 kb, within 900 bp, within 800 bp, within 700 bp, within 600 bp, within 500 bp, within 400 bp, within 300 bp, within 200 bp, or within 100 bp of the repeated sequence.

As noted above, physical linkage of the repeated sequence to the target locus interferes with the ability to quantitatively amplify the target locus. In some embodiments, quantitative PCR is used to quantitatively amplify the locus and amplification of the locus following cleavage from the repeated sequence results in at least an, e.g., 0.5, 1.0, 1.5, 2.0 or more cycle threshold (Ct) difference in accumulation of target locus amplicon compared to a control amplification of the target locus not cleaved from the repeated sequence. Coefficients of variance for replicates of assays measuring DNA methylation content following cleavage from the repeated sequence can be up to 60% lower (e.g., 10, 20, 30, 40, 50% or more lower) than coefficients of variance for replicates of assays measuring DNA methylation content without cleavage from the repeated sequence.

### III. Samples

DNA comprising the target locus to be amplified can be obtained from any biological sample, e.g., from cells, tissues, secretions, and/or fluids from an organism (e.g., an animal, plant, fungus, or prokaryote). The samples may be fresh, frozen, preserved in fixative (e.g., alcohol, formaldehyde, paraffin, or PreServeCyte™) or diluted in a buffer. Biological samples include, e.g., skin, blood or a fraction thereof, tissues, biopsies (from e.g., lung, colon, breast, prostate, cervix, liver, kidney, brain, stomach, esophagus, uterus, testicle, skin, hair, bone, kidney, heart, gall bladder, bladder, and the like), body fluids and secretions (e.g., blood, urine, mucus, sputum, saliva, cervical smear specimens, marrow, feces, sweat, condensed breath, and the like). In some embodiments, the DNA is purified from the biological sample, e.g., from a cell, tissue, or fluid, prior to cleaving the DNA with the restriction enzyme that cleaves between the target locus and the repeated sequence.

In some embodiments, a control sample is provided. A control sample can comprise genomic DNA comprising the target locus of the gene of interest (e.g., FMR-1, FMR-2, DMPK, SCA8, FXN, AR, IT15, DRPLA, SCA1, SCA2, SCA3, SCA6, SCA7, or CSTB) from a normal (i.e., non-diseased) individual or from an individual known to have a disease associated with expanded repeat sequences in that gene. In some embodiments, where the method further comprises detecting and/or quantifying methylation in the target locus, the control sample can comprise DNA comprising the target locus of the gene of interest (e.g., FMR-1, FMR-2, DMPK, SCA8, FXN, AR, IT15, DRPLA, SCA1, SCA2, SCA3, SCA6, SCA7, or CSTB) from a normal (i.e., non-diseased) individual known to not have methylation present at the target locus, or from an individual known to have methylation at the target locus. A control sample can also be an intra-sample control, (i.e., a portion of the biological sample, which is digested with neither a methylation sensitive nor a methylation dependent restriction enzyme, and therefore serves as an uncut control with regards to treatment with a methylation sensitive or a methylation dependent restriction enzyme that would otherwise cut within the locus).

### IV. Cleaving DNA Adjacent to a Repeated Sequence

In one aspect, prior to amplification of a target locus, genomic DNA comprising the target locus adjacent to a repeated sequence is contacted with one or more restriction enzyme that cleaves the genomic DNA between the target locus and the repeated sequence. Generally, in some embodiments, the restriction enzyme(s) does not cleave within the target locus to be amplified.

The restriction enzyme for use in cleaving genomic DNA between a target locus and a repeated sequence can be selected based on a sequence analysis of the region between the target locus and the repeated sequence. In some embodiments, one or more restriction enzymes is selected that cleaves between the target locus and the repeated sequence, but does not cleave within the target locus itself. The sequence analysis can be performed based on evaluating databases of known sequences or in some instances, can be based on empirical determinations, e.g., to take into account variants such as mutations, that may be present in a particular subject.

In some embodiments, the restriction enzyme that cleaves between the target locus and the repeated sequence has a four nucleotide recognition sequence, a six nucleotide recognition sequence, or an eight nucleotide recognition sequence. In some embodiments, the restriction enzyme for use in cleaving genomic DNA between a target locus and a repeated sequence is a methylation-insensitive restriction enzyme, i.e., an enzyme that cuts the DNA regardless of the methylation state of the DNA at or near the enzyme's recognition sequence. In some embodiments, the restriction enzyme is AluI. In some embodiments, the restriction enzyme is not AluI.

Cleavage reactions that cut between the target locus and the repeated sequence will typically be complete digestions. In some embodiments, the cleavage reaction is carried out by incubating the genomic DNA with the restriction enzyme for 45 minutes, 50 minutes, 55 minutes, 60 minutes, 65 minutes, 70 minutes, 75 minutes, 80 minutes, 85 minutes, 90 minutes, or longer. In some embodiments, the cleavage reaction is carried out by incubating the genomic DNA with the restriction enzyme for more than 1 hour. In some embodiments, the cleavage reaction is carried out by incubating the genomic DNA with the restriction enzyme for less than 1 hour.

In some embodiments, the cleavage reaction is stopped by incubating the reaction mixture comprising the genomic DNA and the restriction enzyme by heat inactivation (e.g., at 65° C., at a temperature above 65° C., or for restriction enzymes that cannot be inactivated at 65° C., at 80° C. or at a temperature above 80° C.) for 20 minutes or longer than 20 minutes. In some embodiments, the cleavage reaction is stopped by adding a stop solution (e.g., a solution comprising EDTA or a solution comprising SDS). In some embodiments, the cleavage reaction is stopped by removing the enzyme with a spin column or using phenol/chloroform extraction. Following the cleavage reaction but prior to quantitative amplification, in some embodiments the DNA is purified from the reaction mixture.

### V. Cleaving DNA to Detect Methylation at the Target Locus

In some embodiments, the methods of the present invention further comprise, prior to the quantitative amplification step, contacting the genomic DNA comprising the target locus with one or more methylation state-specific restriction enzymes (e.g., a methylation-sensitive restriction enzyme and/or a methylation-dependent restriction enzyme) to detect and/or quantify methylation at the target locus. The step of cleaving the genomic DNA with the methylation-sensitive restriction enzyme and/or methylation-dependent restriction enzyme can be carried out prior to, concurrent with, or after the step of cleaving the genomic DNA with a restriction enzyme that cleaves between the target locus and the repeated sequence. However the step of cleaving the genomic DNA with the methylation-sensitive restriction enzyme and/or methylation-dependent restriction enzyme is to be carried out prior to the quantitative amplification step.

In some embodiments, for detecting and/or quantifying methylation at the target locus, the genomic DNA sample is divided into at least two (e.g., 2, 3, 4, 5, or more) portions. Different portions can be cut with restriction enzymes having different methylation-sensing activities, as well as one portion acting as an uncut control (although each portion of the genomic DNA sample is treated with the restriction enzyme that cleaves between the target locus and the repeated sequence). Essentially, the methods described in e.g., WO2005/040399; WO 2005/042704; and Holeman et al., Biotechniques 43(5): 683-693 (2007) can be adapted to detect methylation in DNA initially cleaved with the restriction enzyme that cleaves between the target locus and the repeated sequence. In one non-limiting example, a first portion is contacted with a methylation-dependent restriction enzyme (producing intact unmethylated DNA and fragmented methylated DNA) and, optionally a second portion is contacted with a methylation-sensitive restriction enzyme (producing intact methylated DNA and fragmented unmethylated DNA) under conditions in which methylation density can be detected. The intact copies of the locus from each portion are analyzed after the restriction digests (as described below) and compared to each other and/or to intact copies of an uncut portion and/or intact copies in a portion cleaved with both a methylation-dependent restriction enzyme and a methylation-sensitive restriction enzyme.

In one non-limiting example, a first portion is contacted with a methylation-dependent restriction enzyme (producing intact unmethylated DNA and fragmented methylated DNA), a second portion is contacted with a methylation-sensitive restriction enzyme (producing intact methylated DNA and fragmented unmethylated DNA), and a third portion is not digested with a restriction enzyme to provide an analysis of the total number of intact copies of a locus in a sample. The total number of the intact copies of the locus can be compared to the number of methylated loci and/or the number of unmethylated loci to verify that the number of methylated loci and unmethylated loci are equal to the total number of loci.

In still another non-limiting example, a first portion is contacted with a methylation-dependent restriction enzyme (producing intact unmethylated DNA and fragmented methylated DNA), a second portion is contacted with a methylation-sensitive restriction enzyme (producing intact methylated DNA and fragmented unmethylated DNA), a third portion is not digested with a methylation state-specific restriction enzyme, and a fourth portion is digested with both the methylation-sensitive restriction enzyme and the methylation-dependent restriction enzyme and quantitatively amplified. The total number of intact loci remaining after the double digestion can be compared to the number of methylated copies of the locus, unmethylated copies of the locus, and/or total copies of the locus to verify that the number of methylated copies and unmethylated copies are equal to the total number of copies and to verify that the cutting of the methylation sensitive and methylation dependent restriction enzymes is complete.

The order in which the digest(s) are performed are not critical. Thus, although it may be preferable to perform a digest in a certain order, e.g., to first digest with a particular class of methylation-sensing enzymes, e.g., methylation-sensitive enzymes, it is not necessary. Similarly, it may be preferable to perform a double digest. Nor is there a critical order in which to perform the methylation state-specific restriction digest(s) and the restriction digest with the first restriction enzyme that cleaves between the target locus and the repeated sequence.

The amount of undigested DNA in the single digest relative to the double digest and the total number of copies of the locus in the sample is indicative of the proportion of cells that contain unmethylated vs methylated DNA at the locus of interest. Furthermore, such an analysis can serve as a control for the efficacy of the single digest, e.g. the presence of a detectable change in the amount of undigested DNA in the double digest compared to the amount in the single digest with a methylation-sensitive restriction enzyme is an indication that the single digest went to completion.

One of skill in the art will appreciate that, by selecting appropriate combinations of methylation state-specific restriction enzymes (e.g., methylation-sensitive and methylation-dependent restriction enzymes), the methods of the invention can be used to determine cytosine methylation or adenosine methylation at a particular locus based on, e.g., the recognition sequence of the restriction enzyme. For example, by cutting a first portion of a DNA sample comprising the target locus with a methylation-sensitive restriction enzyme which fails to cut when a methylated cytosine residue is in its recognition sequence (e.g., HhaI), and cutting a second portion of DNA sample comprising the target locus with a methylation-dependent restriction enzyme which cuts only if its recognition sequence comprises a methylated cytosine (e.g., McrBC), the cytosine methylation of a particular locus may be determined. Likewise, by cutting a first portion of DNA sample comprising the target locus with a methylation-sensitive restriction enzyme which fails to cut when an adenosine residue is methylated in its recognition sequence (e.g., MboI), and cutting a second portion of DNA sample comprising the target locus with a methylation-dependent restriction enzyme which cuts in the presence of methylated adenosines in its recognition sequence (e.g., DpnI), the adenosine methylation of a particular locus may be determined. In some embodiments, all four sets of digestions are conducted in parallel for both adenosine methylation and cytosine methylation to simultaneously determine the adenosine methylation and the cytosine methylation of a particular locus.

Suitable methylation-dependent restriction enzymes include, e.g., McrBC, McrA, MrrA, DpnI, Msp JI, Lpn PI and Fsp IE. Suitable methylation-sensitive restriction enzymes include restriction enzymes that do not cut when a cytosine within the recognition sequence is methylated at position C⁵ such as, e.g., AatII, Acc65I, AccI, AciI, AclI, AfeI, AgeI, AhdI, AleI, ApaI, ApaLI, AscI, AsiSI, AvaI, AvaII, BaeI, BanI, BbvCI, BbeI, BceI, BcgI, BcoDI, BfuAI, BfuCI, BglI, BmgBI, BsaI, BsaAI, BsaBI, BsaHI, BseYI, BsiEI, BsiWI, BslI, BsmAI, BsmBI, BsmFI, BspDI, BsrBI, BsrFI, BssHII, BssKI, BstAPI, BstBI, BstUI, BstZ17I, BtgZI, Cac8I, ClaI, DpnI, DraIII, DrdI, EaeI, EagI, EarI, EciI, EcoRI, EcoRV, FauI, Fnu4HI, FokI, FseI, FspI, HaeII, HgaI, HhaI, HinP1I, HinfI, HincII, HpaI, HpaII, Hpy166II, Hyp188III, Hpy99I, HpyAV, HpyCH4IV, KasI, MboI, MluI, MapA1I, MmeI, MspI, MspA1I, MwoI, NaeI, NarI, NciI, NheI, NlaIV, NotI, NgoMIV, NruI, Nt.BbvCI, Nt.BsmAI, Nt.CviPII, PaeR7I, PhoI, PleI, PmeI, PmlI, PshAI, PspOMI, PvuI, RsaI, RsrII, SacII, SalI, Sau3AI, Sau96I, ScrFI, SfaNI, SfiI, SfoI, SgrAI, SmaI, SnaBI, StyD4I, TfiI, TseI, TspMI, XmaI, and ZraI. Suitable methylation-sensitive restriction enzymes include restriction enzymes that do not cut when an adenosine within the recognition sequence is methylated at position N⁶ such as, e.g., MboI. One of skill in the art will appreciate that homologs and orthologs of the restriction enzymes described herein are also suitable for use in the present invention.

Either partial or complete restriction enzyme digestions, depending on the methylation state-specific restriction enzyme, can be used to provide information regarding the methylation density within a particular DNA locus. The restriction enzymes for use in the invention are typically selected based on a sequence analysis of the locus of interest. One or more enzymes in each category (e.g., methylation-dependent or methylation-sensitive) are then selected. The sequence analysis can be performed based on evaluating databases of known sequences or in some instances, can be based on empirical determinations, e.g., to take into account variants such as mutations, that may be present in a particular subject.

In some embodiments, the amount of methylation state-specific restriction enzyme (e.g., a methylation-sensitive and/or methylation-dependent restriction enzyme) used results in cleavage such that remaining intact copies are proportional to the amount of methylated or unmethylated copies, depending on which type of restriction enzyme is used. For instance, as shown in Example 9 of WO2005/040399, the amount of enzyme can be tittered such that the amount of enzyme used results in the ability of the enzyme to detect differences in methylation including differences in methylation densities at a locus.

It can be useful to test a variety of conditions (e.g., time of restriction, enzyme concentration, different buffers or other conditions that affect restriction) to identify the optimum set of conditions to resolve subtle or gross differences in methylation density among two or more samples. The conditions may be determined for each sample analyzed or may be determined initially and then the same conditions may be applied to a number of different samples.

### VI. Quantitative Amplification

In some embodiments, the presence and quantity of a target locus adjacent to a repeated sequence can be determined by amplifying the locus following cleavage of the genomic DNA between the target locus and the repeated sequence. Amplification can occur directly after the initial cleavage between the target locus and the repeated sequence, or after one or more additional steps (e.g., cleavage with one or more methylation sensing restriction enzyme as described above). An amplification reaction (e.g., polymerase chain reaction (PCR)) can be designed in which one of the amplification primers binds a nucleotide sequence between the target locus of interest and the cleavage site for the restriction enzyme that cleaves between the target locus and the repeated sequence. With this strategy, because the genomic DNA to be amplified does not comprise the repeated sequence following cleavage with the restriction enzyme, the repeated sequence does not interfere with quantitative amplification, and the quantity of the target locus can accurately determined.

In some embodiments, the presence or absence of methylation at a target locus can also be determined by quantitative amplification. For example, before, during, or after a cleavage reaction with a first restriction enzyme that cleaves between the target locus and the repeated sequence, one or more additional cleavage reactions can be carried out using one or more restriction enzymes that selectively cleave DNA based on the methylation status of the genomic DNA. Because cleavage with the methylation state-specific restriction enzymes (e.g., methylation-sensitive and methylation-dependent restriction enzymes) depends on the methylation state of the DNA, the presence and quantity of methylated target locus can be determined using quantitative amplification. Using a PCR reaction that requires the presence of an intact DNA strand for amplification, the amount of total target locus can be determined by amplifying a DNA sample that has not been contacted with any methylation state-specific restriction enzyme. This amount of total target locus can be compared to the amount of target locus that is amplified from a DNA sample following a cleavage reaction with one or more methylation state-specific restriction enzymes (e.g., a methylation-sensitive restriction enzyme and/or a methylation-dependent restriction enzyme) in order to determine the amount of intact (uncut) DNA that is amplified following methylation-sensitive and/or methylation-dependent cleavage reactions, and thus determine the amount of methylation at the target locus. In these reactions, because a restriction enzyme that cleaves between the target locus and the repeated sequence has also been contacted to the genomic DNA, the genomic DNA to be amplified does not comprise the repeated sequence, and therefore the repeated sequence does not interfere with the quantitative amplification.

A. Quantitative Amplification Methods

Amplification of a DNA locus using reactions is well known (see U.S. Pat. Nos. 4,683,195 and 4,683,202; PCR PROTOCOLS: A GUIDE TO METHODS AND APPLICATIONS (Innis et al., eds, 1990)). Typically, PCR is used to amplify DNA templates. However, alternative methods of amplification have been described and can also be employed, as long as the alternative methods amplify intact DNA to a greater extent than the methods amplify cleaved DNA.

DNA amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology 3:1008-1012 (1985)), allele-specific oligonucleotide (ASO) probe analysis (Conner, et al., PNAS USA 80:278 (1983)), oligonucleotide ligation assays (OLAs) (Landegren, et al., Science 241:1077, (1988)), and the like. Molecular techniques for DNA analysis have been reviewed (Landegren, et al., Science 242:229-237 (1988)).

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol Biotechnol. 20(2):163-79 (2002). Amplifications may be monitored in “real time.”

In general, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., PNAS USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer, Nature Biotech. 14:303-309 (1996), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in hairpin (closed) conformation, the fluorescence of the donor fluorophore is quenched. When employed in PCR, the molecular beacon probe, which hybridizes to one of the strands of the PCR product, is in the open conformation and fluorescence is detected, while those that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of a nucleic acids are also known. For example, some methodologies employ one or more probe oligonucleotides that are structured such that a change in fluorescence is generated when the oligonucleotide(s) is hybridized to a target nucleic acid. For example, one such method involves a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions probes (e.g., Whitcombe et al., Nature Biotechnology 17:804-807, 1999, and U.S. Pat. No. 6,326,145), Sunrise (or Amplifluor) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™).

In other embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In some embodiments, the amount of target locus amplified by quantitative amplification (e.g., qPCR) according to the methods described herein for a genomic DNA sample of interest can be compared to a control sample. For example, in some embodiments, a target locus in a genomic DNA sample of interest from an individual at risk of having a disease associated with an expanded repeat sequence (e.g., fragile X syndrome, FRAXE mental retardation syndrome, myotonic dystrophy, spinocerebellar ataxia type 8, Friedrich's ataxia, spinobulbar muscular atrophy, Huntington's disease, dentatorubralpallidoluysian atrophy, spinocerebellar ataxia type 1, spinocerebellar ataxia type 2, spinocerebellar ataxia type 3, spinocerebellar ataxia type 6, spinocerebellar ataxia type 7, or progressive myoclonus epilepsy of Unverricht-Lundborg type) can be quantified according to the methods of the present invention and compared to a genomic DNA sample comprising said target locus from a normal (i.e., non-diseased) individual in which said expanded repeat sequence is not present.

B. Methylation State-Specific Amplification

In some embodiments, methylation-specific PCR can be employed to monitor the methylation state of specific nucleotides in a DNA locus. In these embodiments, following or preceding digestion with the restriction enzyme, the DNA is combined with an agent that modifies unmethylated cytosines. For example, sodium bisulfite is added to the DNA, thereby converting unmethylated cytosines to uracil, leaving the methylated cytosines intact. One or more primers are designed to distinguish between the methylated and unmethylated sequences that have been treated with sodium bisulfite. For example, primers complementary to the bisulfite-treated methylated sequence will contain guanosines, which are complementary to endogenous cytosines. Primers complementary to the bisulfite-treated unmethylated sequence will contain adenosine, which are complementary to the uracil, the conversion product of unmethylated cytosine. Preferably, nucleotides that distinguish between the converted methylated and unmethylated sequences will be at or near the 3′ end of the primers. Variations of methods using sodium bisulfite-based PCR are described in, e.g., Herman et al., PNAS USA 93:9821-9826 (1996); U.S. Pat. Nos. 5,786,146 and 6,200,756.

C. Detection of Methylation Density

In some embodiments, the methods of the invention can be used to determine the methylation density of a target locus. Determination of methylation density is described, e.g., WO2005/040399; WO 2005/042704; and Holeman et al., Biotechniques 43(5): 683-693 (2007).

The quantity of methylation of a target locus of DNA can be determined by providing a sample of genomic DNA comprising the locus, cleaving the DNA with a restriction enzyme that is either methylation-sensitive or methylation-dependent either before, during, or after cleaving the DNA with a restriction enzyme that cleaves between the target locus and a repeated sequence, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact or cut DNA to a control value representing the quantity of intact or cut DNA in a similarly-treated DNA sample. In some embodiments, the control value can represent a known or predicted number of methylated nucleotides. Alternatively, the control value can represent the quantity of intact or cut DNA from the same target locus from another (e.g., normal, non-diseased) sample, e.g., from a non-diseased individual. Alternatively, the control value can represent the quantity of intact DNA from the same target locus from the same sample, wherein the sample has not been cut with either the methylation sensitive or the methylation dependent restriction enzyme, and therefore the control value represents the total number of copies of the locus in the sample.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus may be determined. If the methylation-sensitive restriction enzyme is contacted to copies of a DNA locus under appropriate conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample.

The average methylation density within a target locus in a DNA sample can be determined by digesting the DNA with a methylation-sensitive or methylation-dependent restriction enzyme and quantifying the relative amount of remaining intact DNA compared to a DNA sample comprising a known amount of methylated DNA.

In some embodiments, the methylation of samples from the same individual is determined over a period of time, e.g., days, weeks, months, or years. Determination of changes in methylation can be useful for providing diagnoses; prognoses; therapy selection; and monitoring progression for various diseases. While the methods of the invention also provide for the detection of specific methylation events, the present methods are particularly notable because they are not limited by a prediction or expectation that the methylation state of a particular nucleotide is determinative of a phenotype. In cases where the density of methylation (rather than the presence or absence of a particular methylated nucleotide) modulates gene expression of a gene characterized by the presence of an expanded repeat sequence, and where the methylation density of a locus reflects disease progression along a continuum, the present methods are particularly helpful.

### VII. Kits

In another aspect, the present invention provides for kits for performing the methods of the invention (e.g., for quantifying a target locus adjacent to a repeated sequence in genomic DNA and/or detecting and/or quantifying methylation at a target locus adjacent to a repeated sequence in genomic DNA). In some embodiments, the kit comprises a restriction enzyme (e.g., a methylation-insensitive restriction enzyme) that cleaves genomic DNA between a target locus and an expanded repeat sequence adjacent to the target locus), and one or more oligonucleotides that specifically amplify the target locus of the gene. In some embodiments, the restriction enzyme and the one or more oligonucleotides are directed to the human gene FMR-1, FMR-2, DMPK, SCAB, FXN, AR, IT15, DRPLA, SCA1, SCA2, SCA3, SCA6, SCA7, or CSTB. In some embodiments, a kit can comprise one or more control nucleic acids comprising the target gene, optionally including the target locus and/or the repeated sequence.

In some embodiments, the kit further comprises one or more restriction enzymes for detecting methylation at a target locus adjacent to an expanded repeat sequence. In some embodiments, the kit further comprises a methylation-dependent restriction enzyme and/or a methylation-sensitive restriction enzyme. In some embodiments, the kit further comprises a control DNA molecule comprising a pre-determined number of methylated nucleotides, and one or more different control oligonucleotide primers that hybridize to the control DNA molecule. In some embodiments, the kits comprise a plurality of DNA molecules comprising different pre-determined numbers of methylated nucleotides to enable the user to compare amplification of a sample to several DNAs comprising a known number of methylated nucleotides.

In some embodiments, the kits of the present invention further comprise written instructions for using the kits. The kits can also comprise reagents sufficient to support the activity of the restriction enzyme(s). The kits can also include a thermostable DNA polymerase.

In some embodiments, the kits may comprise one or more detectably-labeled oligonucleotide probes to monitor amplification of target polynucleotides.

In some embodiments, the kits comprise at least one target oligonucleotide primer that distinguishes between modified unmethylated and methylated DNA in human genomic DNA. In some embodiments, the kits also include an agent that modifies unmethylated cytosine.

In some embodiments, the kits also include a fluorescent moiety that allows the kinetic profile of any amplification reaction to be acquired in real time.

### EXAMPLES

The following examples are offered to illustrate, but not to limit the claimed invention.

### Example 1

### Enzymatic Separation of Amplified Regions of the FMR1 Gene from the CCG Trinucleotide Repeat Region Improves Accuracy and Reproducibility of DNA Methylation Quantification

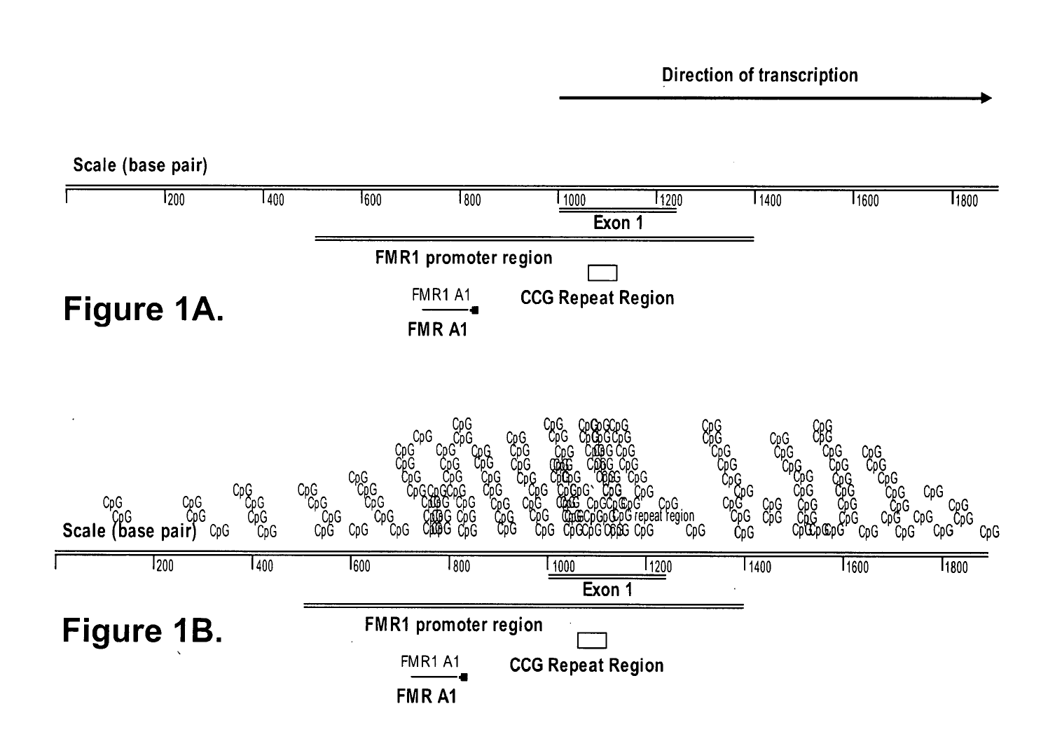

Primers were designed to amplify a 143 base pair amplicon within the promoter region of the FMR1 gene (FMR1 A1, FIG. 1A). The 3′ end of FMR1 A1 is located 162 base pairs upstream of the 5′ start of FMR1 exon 1 and 281 base pairs upstream of the 5′ start of the unstable FMR1 CCG repeat region. The FMR1 A1 amplicon includes seven potential restriction enzyme sites for the methylation sensitive enzyme, Hha I (CGCG) and 13 CpG dinucleotides within potential half sites for recognition by the methylation dependent enzyme, McrBC (Purine-5methylcytosine). Primer sequences are GTCACCGCCCTTCAGCCTTC (SEQ ID NO:1) and GCCCCGCCCTCTCTCTTCAAG (SEQ ID NO:2). The sequence of the amplicon FMR1 A1 is listed as SEQ ID NO:3. The density of CpG dinucleotides within this region is shown in FIG. 1B.

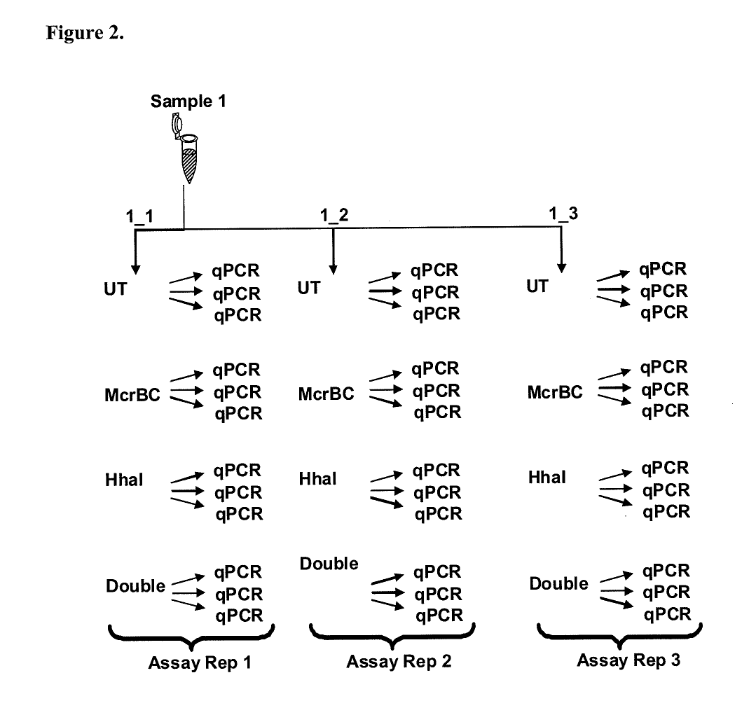

Three genomic DNA samples derived from B cell lymphocytes were analyzed following a triplicated assay strategy in which each assay included three qPCR amplification replicates. The assay design for one B cell sample is diagrammed in FIG. 2. Assay replicate 1 of Sample 1 (1_{—}1; Assay Rep 1 in FIG. 2) included four portions treated as diagrammed in FIG. 2. The first portion (untreated; “UT” in FIG. 2) included 400 ng of genomic DNA, 2 μL of 10× NEB Buffer 2, 0.2 μL, of 100× bovine serum albumin (New England Biolabs), 0.4 μL of 100 mM GTP (Roche), and 0.74 μL of sterile 50% glycerol in a 20 μL total volume. The second portion (methylation-dependent enzyme treated; “McrBC” in FIG. 2) included 400 ng of genomic DNA, 2 μL of 10×NEB Buffer 2, 0.2 μL of 100× bovine serum albumin (New England Biolabs), 0.4 μL of 100 mM GTP (Roche), 0.64 μL of 10 unit/μL McrBC (New England Biolabs), and 0.1 μL sterile 50% glycerol in a 20 μL total volume. The third portion (methylation-sensitive enzyme treated; “HhaI” in FIG. 2) included 400 ng of genomic DNA, 2 μL of 10×NEB Buffer 2, 0.2 μL of 100× bovine serum albumin (New England Biolabs), 0.4 μL of 100 mM GTP (Roche), 0.1 μL of 20 unit/μL Hha I (New England Biolabs), and 0.64 μL sterile 50% glycerol in a 20 μL total volume. The fourth portion (double digest; “Double” in FIG. 2) included 400 ng of genomic DNA, 2 μL of 10×NEB Buffer 2, 0.2 μL of 100× bovine serum albumin (New England Biolabs), 0.4 μL of 100 mM GTP (Roche), 0.64 μL 10 unit/μL McrBC (New England Biolabs) and 0.1 μL of 20 unit/μL Hha I (New England Biolabs) in a 20 μL total volume. Large volume treatment cocktails were made to minimize error from pipetting small volumes. All portions were incubated at 37° C. for 16 hours, followed by incubation at 65° C. for 20 minutes. For each portion, 1 μL of the portion (including 20 ng of DNA) was amplified by qPCR using the FMR1 A1 primers. Each qPCR reaction included, in addition to the 1 μL template portion, 5 μL SYBR Green I Master Mix (Roche) and 2.5 μL primer mixture (2.5 μM) in a total volume of 10 μL. For each portion, three qPCR amplification replicates were performed as diagrammed in FIG. 2. This process was repeated two additional times (“1_{—}2” and “1_{—}3” in FIG. 2) for a total of three independent assay replicates per sample. The entire process diagrammed in FIG. 2 was performed using two additional B cell lymphocyte genomic DNA samples derived from 2 different individuals for a total of three biological replicates. Therefore, each of the three biological replicates included three independent assay replicates of UT, McrBC, Hha I and Double treatments, and each treatment included three qPCR amplification replicates.

Following qPCR analysis, three delta Ct values were calculated, i) McrBC Ct-UT Ct, ii) Hha I Ct-UT Ct, and iii) Double Ct-UT Ct. A theoretical example of calculations based on these Ct values for one assay replicate is shown in Table 1. In this example, the three McrBC-UT delta Ct values are 1.50 cycles (qPCR replicate 1), 1.03 cycles (qPCR replicate 2) and 0.95 cycles (qPCR replicate 3). Delta Ct values for each HhaI-UT and each Double-UT qPCR replicate are also shown. In this example, the McrBC-UT Ct value is greater than the Hha I-UT Ct value for each qPCR replicate. Therefore, the % Sparsely methylated for each replicate is calculated as 2̂−(delta Ct of McrBC-UT). For example, the % Sparse for qPCR replicate 1 is calculated as 2̂−(1.50)=0.3536 (35.4%). The percentage of Intermediately methylated molecules can be calculated if the delta Ct values for both McrBC-UT and Hha I-UT are 1 cycle or greater (i.e. both enzymes are cutting at least 50% of the molecules). In the example shown in Table 1, all Hha I-UT delta Ct values are less than 1 and therefore the sample is reported as having no intermediately methylated molecules. The percentage of molecules that are refractory to digestion with either of the two enzymes is calculated as 2̂−(delta Ct of Double-UT). For example, the % Refractory for qPCR replicate 1 is calculated as 2̂−(3.68)=0.0780 (7.8%). The percentage of densely methylated molecules can therefore be calculated as 100%−% sparse−% intermediate−% refractory. For example, the % Dense for qPCR replicate 1 is calculated as 100%−35.4%−0.00%−7.8%=56.8%.

A second theoretical example of calculations based on these Ct values for one assay replicate is shown in Table 2. In this example, delta Ct values for Hha I-UT are greater than those of McrBC-UT, and no replicates have delta Ct values of 1 cycle or greater for both enzymes.

TABLE 1
Example of Calculations based on McrBC - UT
and Double - UT delta Ct values

| | qPCR Reps | | |
|---|---|---|---|
| | 1 | 2 | 3 |
| McrBC - UT dCt | 1.50 | 1.03 | 1.23 |
| HhaI - UT dCt | 0.00 | 0.30 | 0.00 |
| Double - UT dCt | 3.68 | 6.18 | 5.78 |
| % Dense | 56.8% | 49.6% | 55.5% |
| % Sparse | 35.4% | 49.0% | 42.6% |
| % Intermediate | 0.0% | 0.0% | 0.0% |
| % Refractory | 7.8% | 1.4% | 1.8% |

TABLE 2
Example of Calculations based on Hha I - UT and
Double - UT delta Ct values

| | qPCR Reps | | |
|---|---|---|---|
| | 1 | 2 | 3 |
| McrBC - UT dCt | 0.32 | 0.21 | 0.00 |
| HhaI - UT dCt | 2.58 | 3.68 | 1.89 |
| Double - UT dCt | 3.99 | 4.58 | 5.05 |
| % Dense | 16.7% | 7.8% | 27.0% |
| % Sparse | 77.0% | 86.5% | 100.0% |
| % Intermediate | 0.0% | 0.0% | 0.0% |
| % Refractory | 6.3% | 4.2% | 3.0% |

TABLE 3
Example of Calculations based on McrBC - UT,
Hha I - UT and Double - UT delta Ct values

| | qPCR Reps | | |
|---|---|---|---|
| | 1 | 2 | 3 |
| McrBC - UT dCt | 1.25 | 1.99 | 2.89 |
| HhaI - UT dCt | 2.36 | 1.58 | 1.63 |
| Double - UT dCt | 4.96 | 5.87 | 6.32 |
| % Dense | 19.5% | 33.4% | 32.3% |
| % Sparse | 42.0% | 25.2% | 13.5% |
| % Intermediate | 35.3% | 39.7% | 52.9% |
| % Refractory | 3.2% | 1.7% | 1.3% |

Therefore, the % Densely methylated molecules are calculated as 2̂−(Hha I-UT). For example, the % Dense for replicate 1 is calculated as 2̂−(2.58)=0.1672 (16.7%). In this example shown in Table 2, all McrBC-UT delta Ct values are less than 1 and therefore the sample is reported as having no intermediately methylated molecules. The percentage of molecules that are refractory to digestion with either of the two enzymes is calculated as 2̂−(delta Ct of Double-UT). For example, the % Refractory for replicate 1 is calculated as 2̂−(3.99)=0.0629 (6.3%). The percentage of Sparsely methylated molecules can therefore be calculated as 100%−% dense−% intermediate−% refractory. For example, the % Sparse for qPCR replicate 1 is calculated as 100%−16.7%−0.00%−6.3%=77.0%.

A third theoretical example of calculations based on these Ct values for one assay replicate is shown in Table 3. In this example, delta Ct values for both McrBC-UT and Hha I-UT are 1 cycle or greater. Therefore, the % Sparsely methylated for each replicate is calculated as 2̂−(delta Ct of McrBC-UT). For example the % Sparse for qPCR replicate 1 is calculated as 2̂−(1.25)=0.4204 (42.0%). The % Densely methylated molecules is calculated as 2̂−(Hha I-UT). For example, the % Dense for replicate 1 is calculated as 2̂−(2.36)=0.1948 (19.5%). The percentage of molecules that are refractory to digestion with either of the two enzymes is calculated as 2̂−(delta Ct of Double-UT). For example, the % Refractory for replicate 1 is calculated as 2̂−(4.96)=0.0321 (3.2%). Finally, the % Intermediately methylated molecules is calculated as 100%−% dense−% sparse−% refractory. For example, the % Intermediate for qPCR replicate 1 is calculated as 100%−19.5%−42.0%−3.2%=35.3%.

The results of the standard assay described above on three independent B cell lymphocyte samples are provided in Table 4. For simplicity, the calculated values for Densely methylated molecules only are shown. The calculated average % Dense for each of the three qPCR replicates is shown, as well as the standard deviation (% Dense STDEV) across qPCR replicates and the coefficient of variance (% Dense CV) across qPCR replicates (qPCR Replicate Calculations). Coefficients of variance are calculated as the standard deviation divided by the average. For example, the average % Dense across the three qPCR replicates of sample 1, assay 1 (1_{—}1) is 48.0% with a standard deviation of 2.3% and a coefficient of variance of 4.8%. For the second assay replicate of the same sample (1_{—}2), the average % Dense across the three qPCR replicates is 39.1% with a standard deviation of 22.2% and a coefficient of variance of 56.6%. For the third assay replicate of the same sample (1_{—}3), the average % Dense across the three qPCR replicates is 24.3% with a standard deviation of 5.6% and a coefficient of variance of 23.0%. Calculations for samples 2 (2_{—}1, 2_{—}2, 2_{—}3) and 3 (3_{—}1, 3_{—}2, 3_{—}3) are also shown in Table 4. The average, standard deviation and coefficient of variance across the three assay replicates for each sample are also shown in Table 4 (Assay Replicate Calculations). For example, the average % Dense across the three assays for sample 1 is 37.2%, with a standard deviation of 12.0% and a coefficient of variance of 32.2%. Based on the CV's for qPCR replicates relative to assays replicates, one can conclude that qPCR must contribute to the source of variance of the assay. That is, the CV's between independent assays (including independent treatment portions) are not significantly higher than those between qPCR replicates of the same treatment portions.

TABLE 4
% Densely Methylated Molecules calculations for FMR1 A1 amplicon in three
biological samples.

| | qPCR Replicate Calculations | | | Assay Replicate Calculations | | |
|---|---|---|---|---|---|---|
| Sample_Assay | % Dense | % Dense | % Dense | % Dense | % Dense | % Dense |
| Rep | Average | STDEV | CV | Average | STDEV | CV |
| 1_ 1 | 48.0% | 2.3% | 4.8% | 37.2% | 12.0% | 32.2% |
| 1_2 | 39.1% | 22.2% | 56.6% | | | |
| 1_3 | 24.3% | 5.6% | 23.0% | | | |
| 2_1 | 10.9% | 3.7% | 33.9% | 15.1% | 4.7% | 31.2% |
| 2_2 | 14.1% | 10.5% | 74.7% | | | |
| 2_3 | 20.2% | 10.9% | 54.1% | | | |
| 3_1 | 53.4% | 1.3% | 2.5% | 42.8% | 14.3% | 33.3% |
| 3_2 | 26.6% | 31.5% | 118.3% | | | |
| 3_3 | 48.5% | 27.8% | 57.3% | | | |

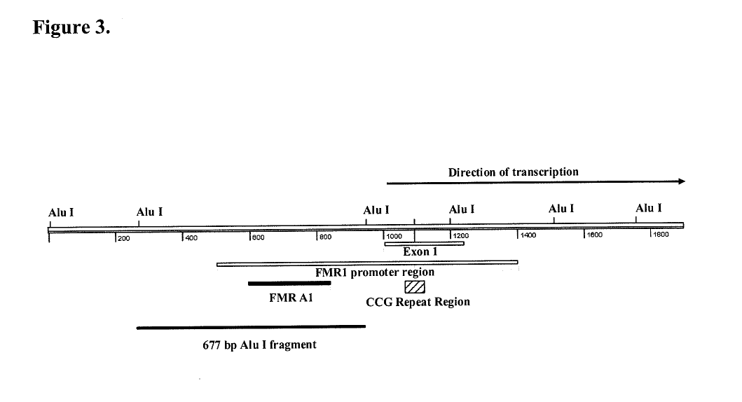

A potential cause of assay variability could be related to the proximity of the amplified region the unstable CGG repeat region of the FMR1 gene. To test this hypothesis, we added 0.4 μL of 10 units/μL Alu I enzyme (New England Biolabs) to each of the treatment portions described in FIG. 2. For example, for assay replicate 1 of sample 1, the Alu I enzyme was added to the UT, McrBC, Hha I and Double portions and these reactions were incubated at 37° C. for 1 hour, followed by incubation at 65° C. for 20 minutes. As shown in FIG. 3, digestion with Alu I results in methylation-independent cutting between the FMR1 A1 amplicon region and the CGG repeat region. Alu I digestion of molecules not previously cut between the two Alu I sites flanking the FMR1 A1 amplicon region would generate a 677 base pair molecule containing the entire FMR1 A1 amplicon region. Therefore, secondary digestion with Alu I results in physically separation of the amplicon region from CCG repeat region. Following this “secondary digest” of all portions, 1 μL of each portion (20 ng DNA) was analyzed by qPCR exactly as described above. All calculations were then performed exactly as described above.

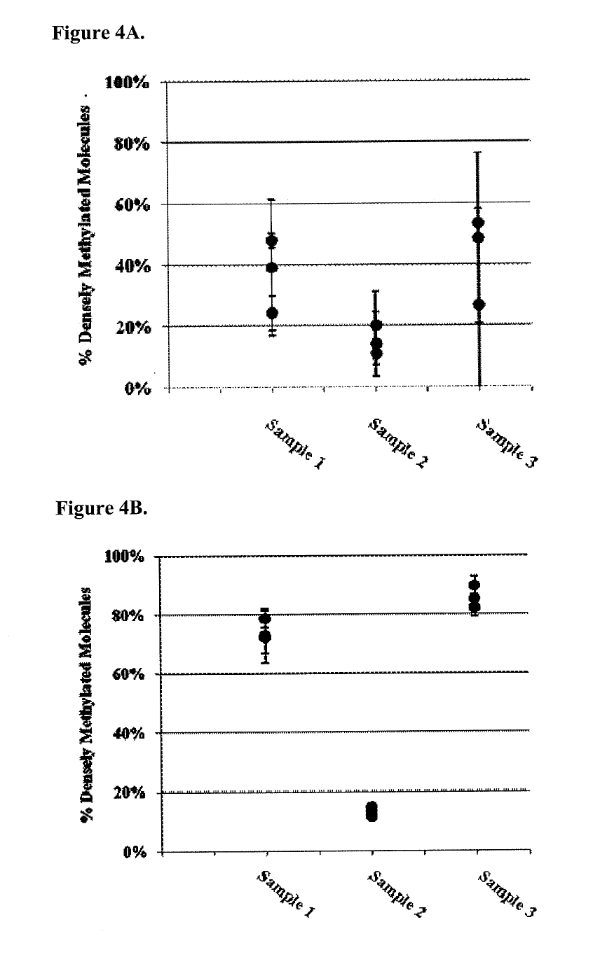

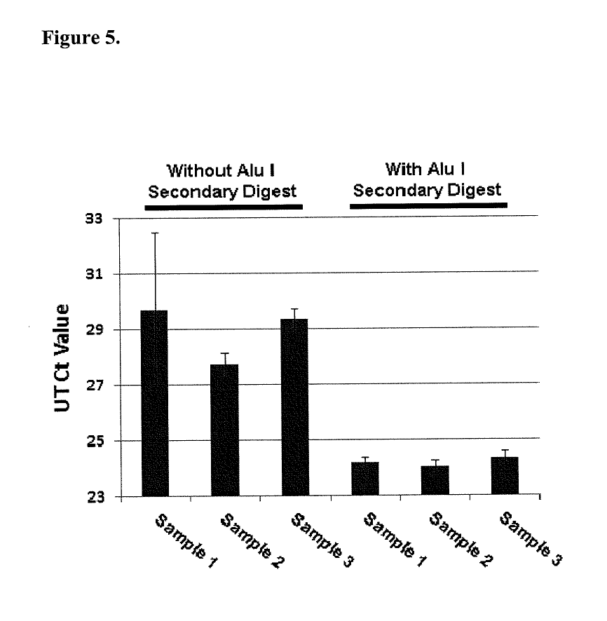

As shown in Table 5, the secondary digest strategy resulted in dramatically reduced standard deviations and coefficients of variance between both qPCR replicates and assay replicates. This effect is shown graphically in FIG. 4. The average % Densely methylated molecules and standard deviations for each qPCR replicate for samples analyzed without the secondary digest are shown in FIG. 4A. The average % Densely methylated molecules and standard deviations for each qPCR replicate for the same samples analyzed with the secondary digest are shown in FIG. 4B. The accuracy and reproducibility of qPCR measures are greater when Ct values are within a lower range (for example, 23 to 25 cycles) than when Ct values are within a higher range (for example, 27 to 30 cycles). FIG. 5 shows the average UT Ct across the nine independent qPCR reactions for the three samples with and without the secondary digest. For each sample, the secondary digest decreases the UT Ct value by at least 3 cycles, representing an approximate 8-fold improvement in amplification efficiency.

TABLE 5
% Densely Methylated Molecules calculations for FMR1 A1 amplicon in three
biological samples following “secondary digest”.

| | qPCR Replicate Calculations | | | Assay Replicate Calculations | | |
|---|---|---|---|---|---|---|
| Sample_Assay | % Dense | % Dense | % Dense | % Dense | % Dense | % Dense |
| Rep | Average | STDEV | CV | Average | STDEV | CV |
| 1_1 | 78.56% | 3.07% | 3.91% | 74.55% | 3.50% | 4.70% |
| 1_2 | 72.97% | 9.27% | 12.71% | | | |
| 1_3 | 72.11% | 5.40% | 7.49% | | | |
| 2 1 | 11.69% | 1.31% | 11.23% | 13.22% | 1.52% | 11.52% |
| 2_2 | 14.73% | 1.31% | 8.87% | | | |
| 2_3 | 13.24% | 2.24% | 16.89% | | | |
| 3_1 | 89.64% | 3.05% | 3.40% | 85.61% | 3.81% | 4.46% |
| 3_2 | 82.06% | 2.72% | 3.32% | | | |
| 3_3 | 85.13% | 3.45% | 4.05% | | | |

Primers were designed to amplify a region of the FMR1 locus that lies downstream of the CCG repeat region. These primers amplify a 215 base pair amplicon (FMR1 A2). The 5′ end of amplicon FMR1 A2 is located 681 bp downstream of the 3′ end of the CCG repeat region. Primer sequences are CACCAAATCACAATGGCAAC (SEQ ID NO:4) and CCATTAGCAGCGCTGCTAC (SEQ ID NO:5). The sequence of amplicon FMR1 A2 is listed as SEQ ID NO:6.

Two samples were analyzed following the strategy diagrammed in FIG. 2, with the exception that the methylation dependent restriction enzyme used was Fsp EI instead of McrBC, and the methylation sensitive restriction enzyme used was Hga I instead of Hha I. The FMR1 A2 amplicon includes 2 Fsp EI sites and 2 Hga I sites. The first portion (Untreated) included 400 ng of genomic DNA, 2 μL of 10×NEB Buffer 4, 0.2 μL of 100× bovine serum albumin (New England Biolabs), and 3 μL of sterile 50% glycerol in a 20 μL, total volume. The second portion (Fsp EI) included 400 ng of genomic DNA, 2 μL of 10×NEB Buffer 4, 0.2 μL of 100× bovine serum albumin (New England Biolabs), 1 μL 10 unit/μL Fsp EI (New England Biolabs) and 2 μL sterile 50% glycerol in a 20 μL total volume. The third portion (Hga I) included 400 ng of genomic DNA, 2 μL of 10×NEB Buffer 4, 0.2 μL of 100× bovine serum albumin (New England Biolabs), 2 μL of 2 units/μL Hga I (New England Biolabs) and 1 μL of sterile 50% glycerol in a 20 μL total volume. The fourth portion (Double) included 400 ng of genomic DNA, 2 μL of 10×NEB Buffer 4, 0.2 of 100× bovine serum albumin (New England Biolabs), 2 μL of 2 units/μL Hga I (New England Biolabs) and 1 μL of 10 unit/μL Fsp EI (New England Biolabs) in a 20 μL total volume. All portions were incubated at 37° C. for 16 hours, followed by incubation at 65° C. for 20 minutes. Each portion was split into two equal 10 μL volume aliquots. One aliquot (secondary digest) was digested with 2 units of the methylation insensitive restriction enzyme Bsa WI (New England Biolabs) at 60° C. for 2 hrs, followed by incubation at 80° C. for 20 minutes to inactivate the enzyme. BSa WI cuts 82 base pairs upstream of the 5′ end of amplicon FMR1 A2. Therefore, secondary digestion with Bsa WI results in physical separation of the amplicon region from CCG repeat region. The second aliquot (without secondary digest) was mock treated without addition of Bsa WI. For each portion, 14 of the portion (including 20 ng of DNA) was amplified by qPCR using the FMR1 A2 primers. Each qPCR reaction included, in addition to the 1 μL template portion, 5 μL SYBR Green I Master Mix (Roche) and 2.5 μL primer mixture (2.5 μM) in a total volume of 10 μL. For each portion, three qPCR amplification replicates were performed as diagrammed in FIG. 2. The DNA samples used were from i) B cell lymphocytes of a male Fragile X Syndrome patient with 501-550 copies of the CCG repeat (Sample 4) and ii) peripheral blood from an unaffected male individual (Sample 5).

As shown in Table 6, the Bsa WI secondary digestion dramatically improves the standard deviation and CV's for both qPCR and Assay replicates of the Fragile X Syndrome B cell lymphocyte sample. The % Dense CV for assay replicates is reduced from 60.2% (without secondary digest) to 0.9% (with secondary). As shown in Table 7, the Bsa WI secondary digestion dramatically improves the standard deviation and CV's for both qPCR and Assay replicates of the unaffected peripheral blood sample. The % Sparse CV for assay replicates is reduced from 53.8% (without secondary) to 1.9% (with secondary).

TABLE 6
DNA Methylation calculations for FMR1 A2 amplicon with and without
secondary Bsa WI digestion.

| | qPCR Replicate | | | Assay Replicate | | |
|---|---|---|---|---|---|---|
| | Calculations | | | Calculations | | |
| | % | % | % | % | % | % |
| Sample_Assay | Dense | Dense | Dense | Dense | Dense | Dense |
| Rep | Average | STDEV | CV | Average | STDEV | CV |
| 4_1 No secondary | 10.0% | 15.6% | 155.3% | 28.7% | 17.3% | 60.2% |
| 4_2 No secondary | 44.1% | 2.9% | 6.6% | | | |
| 4_3 No secondary | 31.9% | 4.1% | 2.8% | | | |
| 4_1 Secondary | 86.9% | 2.1% | 2.5% | 87.2% | 0.8% | 0.9% |
| 4_2 Secondary | 86.5% | 2.0% | 2.4% | | | |
| 4_3 Secondary | 88.1% | 1.0% | 1.1% | | | |

TABLE 7
DNA Methylation calculations for FMR1 A2 amplicon with and without
secondary Bsa WI digestion.

| | qPCR Replicate | | | Assay Replicate | | |
|---|---|---|---|---|---|---|
| | Calculations | | | Calculations | | |
| | % | % | % | % | % | % |
| Sample_Assay | Sparse | Sparse | Sparse | Sparse | Sparse | Sparse |
| Rep | Average | STDEV | CV | Average | STDEV | CV |
| 5_1 No secondary | 10.1% | 7.6% | 75.5% | 25.6% | 13.8% | 53.8% |
| 5_2 No secondary | 36.4% | 6.1% | 16.6% | | | |
| 5_3 No secondary | 30.3% | 15.1% | 49.6% | | | |
| 5_1 Secondary | 73.6% | 3.3% | 4.5% | 72.9% | 1.4% | 1.9% |
| 5_2 Secondary | 73.8% | 5.0% | 6.8% | | | |
| 5_3 Secondary | 71.3% | 6.9% | 9.6% | | | |

### Example 2

### Effect of the Secondary Digest Strategy is Dependent Upon Proximity to the CCG Repeat of FMR1

If the effect of the secondary digest strategy is dependent on the proximity of the amplified region to the CCG repeat region of FMR1, then the effect should decrease as the distance of the amplified region from the CCG repeat region increases. Therefore, five additional primer pairs were designed that amplify regions approximately 1 Kb (amplicon US1), 2 Kb (amplicon US2), 3 Kb (amplicon US3), 4 Kb (amplicon US4) and 5 Kb (amplicon US5) upstream of the CCG repeat region. The US1 primers amplify a 234 base pair amplicon. The 3′ end of US1 is 986 base pair upstream of the 5′ end of the CCG repeat region. Primer sequences for US1 are GGTACTAAGTTCAATGCTGGC (SEQ ID NO:7) and GATGCACCTCCTTGCAACCC (SEQ ID NO:8). The sequence of the US1 amplicon is listed as SEQ ID NO:9. The US2 primers amplify a 279 base pair amplicon. The 3′ end of US2 is 1,952 base pair upstream of the 5′ end of the CCG repeat region. Primer sequences for US2 are GCATACTCGGTAGCAAACTAG (SEQ ID NO:10) and CAGTCCCTACAGGGTCCTTG (SEQ ID NO:11). The sequence of the US2 amplicon is listed as SEQ ID NO:12. The US3 primers amplify a 256 base pair amplicon. The 3′ end of US3 is 2,984 base pair upstream of the 5′ end of the CCG repeat region. Primer sequence for US3 are CATGCACTGGAATGAAGTGAAG (SEQ ID NO:13) and CTTCGCTGGTATCCCTGCAG (SEQ ID NO:14). The sequence of the US3 amplicon is listed as SEQ ID NO:15. The US4 primers amplify a 342 base pair amplicon. The 3′ end of US4 is 3,904 base pair upstream of the 5′ end of the CCG repeat region. Primer sequence for US4 are CTGCTGACACTGTAATGGTGG (SEQ ID NO:16) and CTTTCTTGACACTTTCTCTGAC (SEQ ID NO:17). The sequence of the US4 amplicon is listed as SEQ ID NO:18. The US5 primers amplify a 191 base pair amplicon. The 3′ end of US4 is 4,886 base pair upstream of the 5′ end of the CCG repeat region. Primer sequence for US5 are GACAAGCCACTGTCTGGGAG (SEQ ID NO:19) and GACATATGGCATTGGGCATC (SEQ ID NO:20). The sequence of the US5 amplicon is listed as SEQ ID NO:21.

Because the CpG density of the FMR1 locus is lower in regions farther upstream of the CCG repeat than in regions adjacent to the repeat, the effect of the secondary digest strategy was monitored by analyzing untreated portions only. As shown in FIG. 5, the decrease in the untreated Ct value with the secondary digest relative to without the secondary digest correlates with the improvement in assay reproducibility. To mimic the full assay strategy, each portion included 800 ng of genomic DNA sample, 4 μL 10×NEB Buffer 4, 0.4 μL 100× bovine serum albumin (New England Biolabs) and 0.8 μL sterile 50% glycerol in a 40 μL total volume. All portions were incubated at 37° C. for 16 hours, followed by 65° C. for 20 minutes. Each portion was split into two equal 20 μL volume aliqouts. One aliquot (secondary digest) was digested with 4 units of the methylation insensitive restriction enzyme Bsa WI (New England Biolabs) at 60° C. for 2 hrs, followed by incubation at 80° C. for 20 minutes to inactivate the enzyme. Bsa WI cuts at three sites located between the CCG repeat region and the US1 region. Sites are located approximately 91 base pair, 446 base pair and 533 base pair upstream of the 5′ end of the CCG repeat region. Therefore, secondary digestion with Bsa WI results in physical separation of each of the amplicon regions from CCG repeat region. The second aliquot (without secondary digest) was mock treated without addition of Bsa WI. For each portion, 1 μL of the portion (including 20 ng of DNA) was amplified by qPCR using the each of the five primer pairs separately. The assays were performed on Sample 4 and Sample 5 described in Example 1.

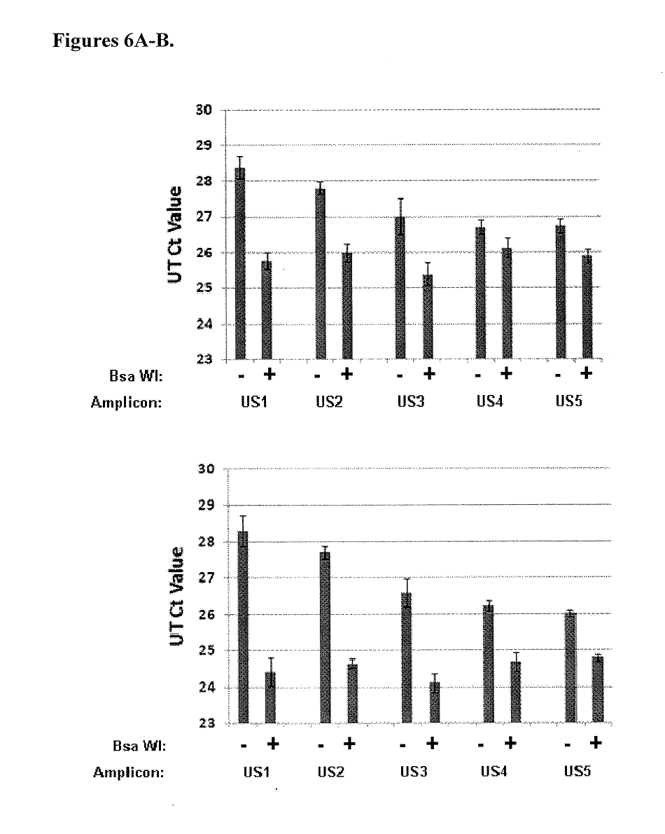

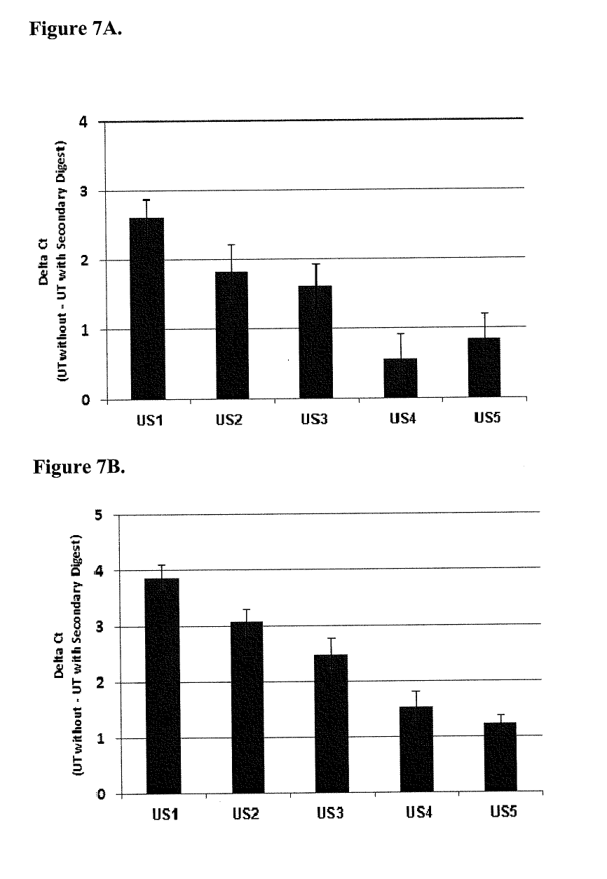

As shown in FIG. 6, the secondary digest strategy decreased the untreated portion Ct value for all five amplicons for both Sample 4 (FIG. 6A) and Sample 5 (FIG. 6B). Each bar represents the average of 9 qPCR amplifications (3 qPCR replicates of each of 3 treatment replicates). However, the degree of this improvement decreases as the distance between the amplicon and the CCG repeat region increases. For example, in Sample 4, the secondary digest strategy decreases the untreated Ct value of the US1 amplicon from 28.4 (without secondary digest) to 25.8 (with secondary digest), representing an approximate 6-fold improvement in amplification. However, in the same Sample, the secondary digest strategy decreases the untreated Ct value of the US5 amplicon from 26.7 (without secondary digest) to 25.9 (with secondary digest), representing an approximate 2-fold improvement in amplification. FIG. 7 demonstrates the decrease in the delta Ct value of the untreated sample without secondary digest minus the Ct value of the untreated sample with secondary digest for Sample 4 (FIG. 7A) and Sample 5 (FIG. 7B). Therefore, the effect of the secondary digest strategy is dependent upon the proximity of the amplicon to the CCG repeat region.

### Example 3

### Enzymatic Separation of an Amplified Region of the DMPK Gene from the CTG Trinucleotide Repeat Region Improves Accuracy and Reproducibility of DNA Methylation Quantification

Based on the data above, the effect of the secondary digest strategy could have been exclusive to the FMR1 CCG repeat region or could represent a more general effect related to other trinucleotide repeat classes. To obtain insight into these possibilities, primers were designed to analyze DNA methylation near the CTG repeat of the DMPK gene. Trinucleotide repeat expansions of the CTG repeat located in the 3′ untranslated region of the DMPK gene are responsible for Myotonic Dystrophy Type 1 (DM1). The number of CTG repeat units in unaffected individual varies, but the average number is below 40 units. In affected individuals, the repeat length can range from approximately 50 units to greater than 4,000 units.

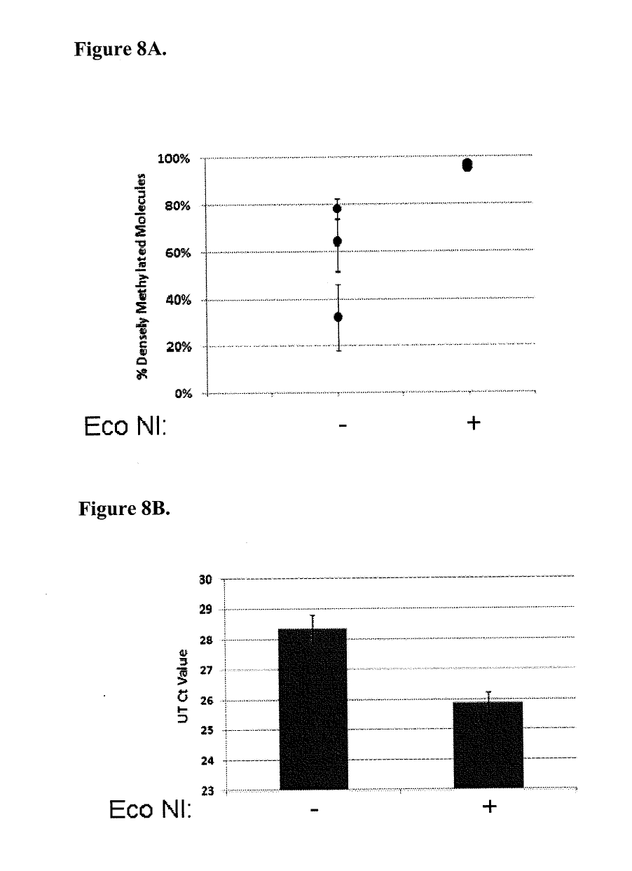

Primers were designed to amplify a 165 base pair amplicon upstream of the DMPK gene CTG repeat. The 3′ end of this amplicon (DMPK A1) is 391 base pair upstream of the 5′ end of the DMPK CTG repeat. The amplicon includes one potential restriction enzyme site for the methylation sensitive enzyme, Hha I (CGCG) and 11 CpG dinucleotides within potential half sites for recognition by the methylation dependent enzyme, McrBC (Purine-5methylcytosine). Primer sequences are GCCAATGACGAGTTCGGACG (SEQ ID NO:22) and GAGGTCAACACCCGGCATG (SEQ ID NO:23). The sequence of the DMPK A1 amplicon is listed as SEQ ID NO:24. Analysis followed the same qPCR and assay replicate strategy diagrammed in FIG. 2. The genomic DNA sample (Sample 6) was derived from B cell lymphocytes of a DM1 affected male individual. The expanded repeat allele included greater than 2,000 units of the CTG repeat. The first portion (untreated; “UT” in FIG. 2) included 200 ng of genomic DNA, 1 μL of 10×NEB Buffer 2, 0.1 μL of 100× bovine serum albumin (New England Biolabs), 0.2 μL of 100 mM GTP (Roche), and 0.5 μL of sterile 50% glycerol in a 10 μL total volume. The second portion (methylation-dependent enzyme treated; “McrBC” in FIG. 2) included 200 ng of genomic DNA, 1 μL of 10×NEB Buffer 2, 0.1 μL of 100× bovine serum albumin (New England Biolabs), 0.2 μL of 100 mM GTP (Roche), 0.32 μL of 10 unit/μL McrBC (New England Biolabs), and 0.18 μL sterile 50% glycerol in a 10 μL total volume. The third portion (methylation-sensitive enzyme treated; “HhaI” in FIG. 2) included 200 ng of genomic DNA, 1 μL of 10×NEB Buffer 2, 0.1 μL of 100× bovine serum albumin (New England Biolabs), 0.2 μL of 100 mM GTP (Roche), 0.05 μL of 20 unit/μL Hha I (New England Biolabs), and 0.47 μL sterile 50% glycerol in a 20 μL total volume. The fourth portion (double digest; “Double” in FIG. 2) included 200 ng of genomic DNA, 1 μL of 10×NEB Buffer 2, 0.1 μL of 100× bovine serum albumin (New England Biolabs), 0.2 μL of 100 mM GTP (Roche), 0.32 μL 10 unit/μL McrBC (New England Biolabs) and 0.05 μL of 20 unit/μL Hha I (New England Biolabs) in a 20 μL total volume. Large volume treatment cocktails were made to minimize error from pipetting small volumes. All portions were incubated at 37° C. for 16 hours, followed by incubation at 65° C. for 20 minutes. Each portion was then split into 2 equal 5 μL aliquots. One aliquot was digested with 2 units of the DNA methylation insensitive restriction enzyme Eco NI (New England Biolabs) at 37° C. for one hour, followed by incubation at 65° C. for 20 minutes to inactivate the enzyme. Eco NI cuts at a site 357 bp upstream of the 5′ end of the CTG repeat region. Therefore, digestion with Eco NI physically separates the amplified region from the CTG repeat region. For each portion, 1 μL of the portion (including 20 ng of DNA) was amplified by qPCR using the DMPK A1 primers. Each qPCR reaction included, in addition to the 1 μL template portion, 5 μL SYBR Green I Master Mix (Roche) and 2.5 μL primer mixture (2.5 μM) in a total volume of 10 μL. For each portion, three qPCR amplification replicates were performed and three treatment condition replicates were performed as diagrammed in FIG. 2. DNA methylation calculations were performed exactly as described in Example 1. As shown in Table 8, the secondary digest strategy dramatically improves both the qPCR and assay replicate reproducibility. For example, the CV of assay replicates was reduced from 40.5% (without secondary digest) to 0.8% (with secondary digest). The affect on assay reproducibility is shown graphically in FIG. 8A and on the untreated portion Ct value in FIG. 8B. Therefore, the benefit of the secondary digest strategy is not limited to the FMR1 locus or on proximity to a CCG:CGG repeat, but is also relevant to the DMPK locus and proximity to a CTG:CAG repeat.

TABLE 8
Secondary Digest Strategy improves assay reproducibility at the DMPK
locus.

| | qPCR Replicate | | | Assay Replicate | | |
|---|---|---|---|---|---|---|
| | Calculations | | | Calculations | | |
| | % | % | % | % | % | % |
| Sample_Assay | Dense | Dense | Dense | Dense | Dense | Dense |
| Rep | Average | STDEV | CV | Average | STDEV | CV |
| 6_1 No Secondary | 78.2% | 4.3% | 5.5% | 58.2% | 23.6% | 40.5% |
| 6_2 No Secondary | 32.2% | 14.0% | 43.5% | | | |
| 6_3 No Secondary | 64.3% | 12.8% | 20.0% | | | |
| 6_1 Secondary | 96.7% | 1.0% | 1.0% | 96.4% | 0.8% | 0.8% |
| 6_2 Secondary | 95.5% | 1.6% | 1.7% | | | |
| 6_3 Secondary | 97.1% | 0.4% | 0.4% | | | |

### Example 4

### Enzymatic Separation of an Amplified Region of FXN Gene from the GAA Trinucleotide Repeat Region has No Effect on Accuracy and Reproducibility of DNA Methylation Quantification

The trinucleotide repeats associated with the FMR1 and DMPK genes are high G+C content and may be expected to form stable secondary DNA structures that might interfere with efficient quantitative amplification of neighboring loci. To test whether the effect of the secondary digest strategy is dependent upon G+C content of the repeat, primers were designed to analyze DNA methylation content near the GAA repeat of the FXN gene. Expansions of the GAA repeat in the first intron of the FXN gene are responsible for Friedrich Ataxi (FRDA). Unaffected alleles contain less than approximately 38 repeat units, while expanded alleles in affected individuals range from approximately 70 units to over 1,000 units.

Primers were designed to amplify a 319 base pair amplicon (FXN A1) upstream of the GAA repeat region. The 3′ end of the FXN A1 amplicon is 170 base pair upstream of the 5′ end of the GAA repeat region. The amplicon includes two potential restriction enzyme sites for the methylation sensitive enzyme, Aci I (CCGC) and 7 CpG dinucleotides within potential half sites for recognition by the methylation dependent enzyme, McrBC (Purine-5methylcytosine). Primer sequences are GAATGGCTGTGGGGATGAGG (SEQ ID NO:25) and CTTTTAAGCACTGGCAACCAATC (SEQ ID NO:26). The sequence of the FXN A1 amplicon is listed as SEQ ID NO:27. Analysis followed the same qPCR and assay replicate strategy diagrammed in FIG. 2, except that Aci I was used as the methylation sensitive restriction enzyme instead of Hha I. The genomic DNA samples analyzed were derived from B cell lymphocytes of a FRDA affected male individual with on allele of FXN including 650 AAG repeat units and the other allele including 1030 repeat units (Sample 7) and from B cell lymphocytes of an unaffected male individual (Sample 8). The first portion (untreated; “UT” in FIG. 2) included 800 ng of genomic DNA, 44 of 10×NEB Buffer 2, 0.4 μL of 100× bovine serum albumin (New England Biolabs), 0.8 μL of 100 mM GTP (Roche), and 1.1 μL of sterile 50% glycerol in a 40 μL total volume. The second portion included 800 ng of genomic DNA, 4 μL of 10×NEB Buffer 2, 0.4 μL of 100× bovine serum albumin (New England Biolabs), 0.8 μL of 100 mM GTP (Roche), 1.3 μL of 10 unit/μL McrBC (New England Biolabs), and 0.08 μL sterile 50% glycerol in a 40 μL total volume. The third portion included 800 ng of genomic DNA, 4 μL of 10×NEB Buffer 2, 0.4 μL of 100× bovine serum albumin (New England Biolabs), 0.8 μL of 100 mM GTP (Roche), 0.8 μL of 10 unit/μL Aci I (New England Biolabs), and 1.3 μL sterile 50% glycerol in a 40 μL total volume. The fourth portion included 800 ng of genomic DNA, 4 μL of 10×NEB Buffer 2, 0.4 μL, of 100× bovine serum albumin (New England Biolabs), 0.8 μL of 100 mM GTP (Roche), 1.3 μL 10 unit/μL McrBC (New England Biolabs) and 0.8 μL of 10 unit/μL Aci I (New England Biolabs) in a 40 μL total volume. Large volume treatment cocktails were made to minimize error from pipetting small volumes. All portions were incubated at 37° C. for 16 hours, followed by incubation at 65° C. for 20 minutes. Each portion was then split into 2 equal 20 μL aliquots. One aliquot was digested with 4 units of the DNA methylation insensitive restriction enzyme Avr II (New England Biolabs) at 37° C. for one hour, followed by incubation at 80° C. for 20 minutes to inactivate the enzyme. Avr II cuts at a site 98 bp upstream of the 5′ end of the GAA repeat region. Therefore, digestion with Avr II physically separates the amplified region from the GAA repeat region. For each portion, 1 μL of the portion (including 20 ng of DNA) was amplified by qPCR using the FXN A1 primers. Each qPCR reaction included, in addition to the 1 μL template portion, 5 μL SYBR Green I Master Mix (Roche) and 2.5 μL primer mixture (2.5 μM) in a total volume of 10 μL. For each portion, three qPCR amplification replicates were performed and three treatment condition replicates were performed as diagrammed in FIG. 2. DNA methylation calculations were performed exactly as described in Example 1.

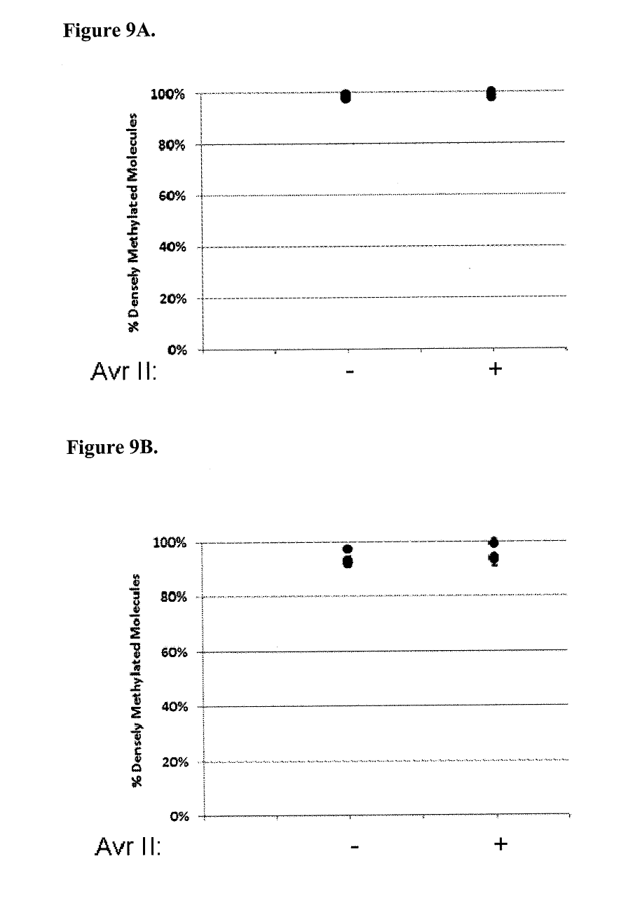

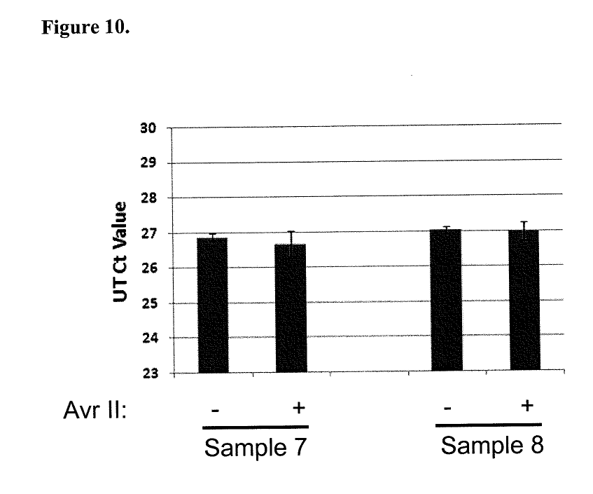

As shown in Table 9, the secondary digest strategy had no significant effect on the reproducibility of DNA methylation measurements at the FXN locus. For example, the CV of assay replicates are all below 5%, regardless of whether the secondary digest strategy was used. These results are shown graphically in FIG. 9 for Sample 7 (FIG. 9A) and Sample 8 (FIG. 9B). Likewise, the secondary digest strategy did not affect the Ct value of the untreated qPCR reactions (FIG. 10). These results suggest that the effect of the secondary digest is related to the proximity to trinucleotide repeat classes that are expected to form stable secondary DNA structures that could interfere with efficient and accurate quantitative amplification.

TABLE 9
Secondary Digest Strategy has no effect on reproducibility at the FXN
locus.

| | qPCR Replicate | | | Assay Replicate | | |
|---|---|---|---|---|---|---|
| | Calculations | | | Calculations | | |
| | % | % | % | % | % | % |
| Sample_Assay | Dense | Dense | Dense | Dense | Dense | Dense |
| Rep | Average | STDEV | CV | Average | STDEV | CV |
| 7_1 No Secondary | 97.8% | 0.3% | 0.3% | 98.5% | 0.7% | 0.7% |
| 7_2 No Secondary | 98.4% | 0.2% | 0.2% | | | |
| 7_3 No Secondary | 99.3% | 0.4% | 0.4% | | | |
| 7_1 Secondary | 98.4% | 0.8% | 0.8% | 99.1% | 0.8% | 0.8% |
| 7_2 Secondary | 98.9% | 0.5% | 0.5% | | | |
| 7_3 Secondary | 99.9% | 0.1% | 0.1% | | | |
| 8_1 No Secondary | 92.3% | 1.1% | 1.2% | 94.3% | 2.7% | 2.9% |
| 8_2 No Secondary | 93.3% | 1.7% | 1.9% | | | |
| 8_3 No Secondary | 97.4% | 0.6% | 0.6% | | | |
| 8_1 Secondary | 93.2% | 2.2% | 2.4% | 95.5% | 3.3% | 3.4% |
| 8_2 Secondary | 94.1% | 1.0% | 1.1% | | | |
| 8_3 Secondary | 99.3% | 0.1% | 0.1% | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of quantifying a target locus adjacent to a repeated sequence in genomic DNA, wherein proximity to the repeated sequence interferes with amplification of the target locus, the method comprising:
cleaving the genomic DNA with a first restriction enzyme between the target locus and the repeated sequence, wherein the cleaving does not cleave the target locus; and
quantitatively amplifying the target locus.

2. The method of claim 1, wherein the target locus is at least 100, 200, 300, 400, or 500 base pairs long.

3. The method of claim 1, wherein the quantitative amplifying comprises quantitative polymerase chain reaction (qPCR).

4. The method of claim 1, wherein the repeated sequence comprises a continuous repetition of a motif of two, three, four, or five base pairs repeated at least ten times.

5. The method of claim 1, wherein the repeated sequence comprises a continuous repetition of a motif of three base pairs repeated at least ten times.

6. (canceled)

7. The method of claim 1, wherein the repeated sequence is selected from the group consisting of CAG, CTG, CCG, CGG, GAC, GTC, GGC, GCC, GCG, ACG, TCG, CGA, CGT, CGC, GCA, GCT, GCC, TGC, AGC and CA.

8. The method of claim 1, wherein the repeated sequence and the target locus are less than 10 kb apart.

9. The method of claim 1, wherein the genomic DNA comprises human DNA.

10. The method of claim 1, wherein the target locus is a promoter region.

11. The method of claim 9, wherein the target locus is within the human Fragile Mental Retardation-1 (FMR-1); Fragile Mental Retardation-2 (FMR-2); dystrophia myotonica protein kinase (DMPK); spinocerebellar ataxia type 8 (SCAB); androgen receptor (AR); huntingtin (IT15); dentatorubralpallidoluysian atrophy (DRPLA); spinocerebellar ataxia type 1 (SCA1); spinocerebellar ataxia type 2 (SCA2); spinocerebellar ataxia type 3 (SCA3); spinocerebellar ataxia type 6 (SCA6); spinocerebellar ataxia type 7 (SCA7); or cystatin B (CSTB) gene.

12. The method of claim 1, wherein the first restriction enzyme is a methylation-insensitive restriction enzyme.

13. The method of claim 1, further comprising contacting the DNA, before, concurrent with, or after the cleaving, but before the quantitative amplifying, with at least one more restriction enzyme.

14. The method of claim 13, wherein the at least one more restriction enzyme is a methylation-dependent restriction enzyme or a methylation-sensitive restriction enzyme.

15. The method of claim 14, wherein the methylation-dependent restriction enzyme or the methylation-sensitive restriction enzyme are used under conditions such that the amount of remaining intact copies of the locus is inversely proportional or directly proportional, respectively, to the methylation density of the locus.

16. The method of claim 14, comprising contacting the DNA with a methylation-dependent restriction enzyme or a methylation-sensitive restriction enzyme, before, concurrent with, or after cleaving with a first restriction enzyme, but before the quantitative amplification step.

17. (canceled)

18. The method of claim 1, wherein the first restriction enzyme is not AluI.

19. The method of claim 13, wherein the DNA is cleaved with the first restriction enzyme for more than 1 hour.

20. A method of detecting the quantity of methylation at a target locus adjacent to a repeated sequence in a genomic DNA sample, wherein proximity to the repeated sequence interferes with amplification of the target locus, the method comprising:
(a) cleaving the genomic DNA sample with a first restriction enzyme between the target locus and the repeated sequence, wherein the cleaving does not cleave the target locus;
(b) dividing the genomic DNA sample into at least two physically distinct portions, thereby generating a first portion and a second portion;
(c) contacting the first portion with a methylation-sensitive restriction enzyme to obtain a genomic DNA sample comprising fragmented unmethylated copies of the target locus and intact methylated copies of the target locus;
(d) quantifying the number of intact copies of the target locus in the first portion by quantitative amplification;
(e) contacting the second portion with a methylation-dependent restriction enzyme to obtain a genomic DNA sample comprising fragmented methylated copies of the target locus and intact unmethylated copies of the target locus;
(f) quantifying the number of intact copies of the target locus in the second portion by quantitative amplification; and
(g) determining the quantity of methylation at the target locus by comparing the number of intact copies of the target locus in the first portion and the number of intact copies of the target locus in the second portion.

21.-32. (canceled)

33. A kit for quantifying a target locus adjacent to a repeated sequence, the kit comprising:
(ii) one or more oligonucleotides that specifically amplify the target locus of the gene; and
(iii) a restriction enzyme that cleaves human genomic DNA between the repeated sequence and the target locus but does not cleave the target locus.

34.-37. (canceled)

38. The kit of claim 33, further comprising reagents for detecting the amplification of the target locus by quantitative polymerase chain reaction (qPCR).
